(19) United States
(12) Patent Application Publication
(10) Pub. No.: US 2020/0017888 A1
(43) Pub. Date: Jan. 16, 2020

(54) SYNTHETIC METHYLOTROPHS AND USES THEREOF

(71) Applicants: Eleftherios T. Papoutsaki, Newark, DE (US); Robert Kyle Bennett, Elkton, MD (US); Jacqueline Gonzalez, Elkton, MD (US); William Brian Whitaker, Wilmington, DE (US); Maciek Antoniewicz, Baltimore, MD (US)

(72) Inventors: Eleftherios T. Papoutsaki, Newark, DE (US); Robert Kyle Bennett, Elkton, MD (US); Jacqueline Gonzalez, Elkton, MD (US); William Brian Whitaker, Wilmington, DE (US); Maciek Antoniewicz, Baltimore, MD (US)

(73) Assignee: University of Delaware, Newark, DE (US)

(21) Appl. No.: 16/519,070

(22) PCT Filed: Feb. 13, 2018
(86) PCT No.: PCT/US2018/017913

§ 371 Date: Jul. 23, 2019

Related U.S. Application Data
(60) Provisional application No. 62/458,053, filed on Feb. 13, 2017.

(51) Int. Cl. C12P 7/28; C12N 9/10; C12N 9/88; C07K 14/245

(52) U.S. Cl. CPC C12P 7/28; C12N 9/1029; C12N 9/13; C07K 14/245; C12Y 203/01009; C12Y 208/03009; C12Y 401/01004; C12N 9/88

(57) ABSTRACT

The present invention provides a method for increasing production of a metabolite by a non-naturally occurring methylotroph, comprising growing the non-naturally occurring methylotroph in a medium comprising methanol. Expression of one or more native genes in the non-naturally occurring methylotroph is changed. Also provided are the non-naturally occurring methylotroph and preparation thereof.

### CROSS-REFERENCE TO RELATED APPLICATION

This application claims priority to U.S. Provisional Application No. 62/458,053, filed Feb. 13, 2017, the entire disclosure of which is incorporated herein by reference in its entirety for all purposes.

### REFERENCE TO U.S. GOVERNMENT SUPPORT

This invention was made with government support under Grant No. DE-AR0000432 awarded by the U.S. Advanced Research Projects Agency-Energy (ARPA-E) of Department of Energy (DOE). The United States has certain rights in the invention.

### FIELD OF THE INVENTION

The invention relates generally to non-naturally occurring methylotrophs and uses thereof, especially for production of metabolites.

### BACKGROUND OF THE INVENTION

Natural gas consists primarily of methane (CH₄), and includes smaller amounts of higher alkanes, CO₂, N₂, and H₂S. It is used not only for heating and energy generation, but also as a chemical feedstock to produce commodity chemicals that can be then converted to plastics and specialty chemicals. Natural gas constitutes an enormous energy and chemical resource for the U.S. where the recoverable amount is estimated to be 2,000 trillion ft³. Natural gas is however a poor transportation fuel because of its inherently low energy density. Technologies that can convert natural gas into liquid fuels at competitive prices will not only lessen our dependence on imported oil, but also eliminate the needs for retrofitting existing transportation infrastructure. Current chemical routes based on chemical conversion to syngas (CO & H₂) through the Fischer-Tropsch process are not competitive for producing liquid fuels, as they suffer from both high capital costs and low conversion efficiencies. Bioconversion is a promising alternative because of its high specificity and high process energy efficiency all under very mild conditions. Thus, CH₄ represents an ideal target for conversion to liquid fuels by biological processes or hybrid biological/catalytic processes.

Some progress has been made in the catalytic conversion of CH₄ to methanol (MeOH), and more biological means may be developed for converting methane to methanol. There remains a need for non-naturally occurring (i.e., synthetic or recombinant) methylotrophic microbes (also referred to as microorganisms) capable of converting methanol efficiently to liquid fuel molecules or other commodity chemicals.

### SUMMARY OF THE INVENTION

The present invention relates to non-naturally occurring methylotrophs and uses thereof.

The present invention provides a method for increasing production of a metabolite by a non-naturally occurring methylotroph. The method comprises growing the non-naturally occurring methylotroph in a medium comprising methanol. The methanol may be either supplied directly to the medium or produced from methane supplied to the medium by the action of soluble methane monooxygenase (sMMO) expressed in the non-naturally occurring methylotroph. Expression of one or more native genes in the non-naturally occurring methylotroph is changed. The one or more native genes may comprise one or more deletions.

According to the method of the present invention, the one or more native genes may comprise 2,3-bisphosphoglycerate-dependent phosphoglycerate mutase gene (gpmA), 2,3-bisphosphoglycerate-independent phosphoglycerate mutase gene (gpmM), 2-dehydro-3-deoxy-D-gluconate 6-phosphate (KDPG) aldolase gene (eda), 6-phosphogluconate dehydrogenase gene (gnd), aminomethyltransferase gene (gcvT), bifunctional (p)ppGpp synthetase II/guanosine-3′,5′-bis pyrophosphate 3′-pyrophosphohydrolase gene (spoT), enolase gene (eno), fructose-1,6-bisphosphatase 1 class 2 gene (glpX), fructose-1,6-bisphosphatase class 1 gene (fbp), GDP pyrophosphokinase/GTP pyrophosphokinase gene (relA), glucose-6-phosphate isomerase gene (pgi), glyceraldehyde-3-phosphate dehydrogenase gene (gapA), glyceraldehyde-3-phosphate dehydrogenase gene (gapC), glycine cleavage system H protein gene (gcvH), glycine decarboxylase gene (gcvP), HTH-type transcriptional regulator GntR gene (gntR), leucine-responsive regulatory protein gene (lrp), methylglyoxal synthase gene (mgsA), phosphogluconate dehydratase gene (edd), phosphoglycerate dehydrogenase gene (serA), phosphoglycerate kinase gene (pgk), ribulose-phosphate 3-epimerase gene (rpe), RNA polymerase-binding transcription factor gene (dksA), serine hydroxymethyltransferase gene (glyA), transaldolase A gene (talA), transaldolase B gene (talB), or a combination thereof. In one embodiment, the one or more native genes are deleted from the genome of the non-naturally occurring methylotroph and comprise leucine-responsive regulatory protein gene (lrp).

The medium may further comprise a co-substrate. The co-substrate may comprise one or more monosaccharides selected from the group consisting of glucose, xylose, mannose, arabinose, rhamnose, ribose and a combination thereof. The co-substrate may comprise one or more amino acids selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine and a combination thereof.

The non-naturally occurring methylotroph may be a microbe derived from a microbe selected from the group consisting of Acetobacter, Acinetobacter, Bacillus, Chlorobi, Clostridium, Corynebacterium, Cyanobacteria, Deinococcus, Enterobacter, Enterobacteria, Escherichia, Geobacillus, Geobacter, Klebsiella, Lactobacillus, Lactococcus, Mannheimia, Propionibacterium, Pseudomonas, Ralstonia, Shewanella, Streptococcus, Streptomyces, Synechococcus, Synechocystis and Zymomonas. The non-naturally occurring methylotroph may be Escherichia coli.

The method may further comprise incorporating a carbon atom from the methanol into the metabolite.

The metabolite may be selected from the group consisting of 4-carbon chemicals, diacids, 3-carbon chemicals, higher carboxylic acids (e.g., acetic acid, propionic acid, butyric acid, valeric (pentanoic) acid and caproic (hexanoic) acid), alcohols of higher carboxylic acids and polyhydroxyalkanoates. The metabolite may be selected from the group consisting of acetone, isopropanol, 1,3-butanediol and n-butanol.

Where the metabolite is acetone, the method may further comprise expressing a heterologous enzyme having an acetyl-CoA C-acetyltransferase activity (e.g., thiolase (THL, EC 2.3.1.9)), a heterologous enzyme having a butyrate-acetoacetate CoA-transferase activity (e.g., acyl(acetate/butyrate)-acetoacetate coenzyme A transferase (CTFA/B, EC 2.8.3.9)), and a heterologous enzyme having acetoacetate decarboxylase activity (e.g., acetoacetate decarboxylase (ADC, EC 4.1.1.4)) in the non-naturally occurring methylotroph.

The method may further comprise increasing the production of the metabolite by the non-naturally occurring methylotroph by at least about 5% as compared with that by a control microbe in which the expression of the one or more native genes is not changed.

The present invention also provides a non-naturally occurring methylotroph in which expression of one or more native genes is changed. The one or more native genes may comprise 2,3-bisphosphoglycerate-dependent phosphoglycerate mutase gene (gpmA), 2,3-bisphosphoglycerate-independent phosphoglycerate mutase gene (gpmM), 2-dehydro-3-deoxy-D-gluconate 6-phosphate (KDPG) aldolase gene (eda), 6-phosphogluconate dehydrogenase gene (gnd), aminomethyltransferase gene (gcvT), bifunctional (p)ppGpp synthetase II/guanosine-3′,5′-bis pyrophosphate 3′-pyrophosphohydrolase gene (spoT), enolase gene (eno), fructose-1,6-bisphosphatase 1 class 2 gene (glpX), fructose-1,6-bisphosphatase class 1 gene (fbp), GDP pyrophosphokinase/GTP pyrophosphokinase gene (relA), glucose-6-phosphate isomerase gene (pgi), glyceraldehyde-3-phosphate dehydrogenase gene (gapA), glyceraldehyde-3-phosphate dehydrogenase gene (gapC), glycine cleavage system H protein gene (gcvH), glycine decarboxylase gene (gcvP), HTH-type transcriptional regulator GntR gene (gntR), leucine-responsive regulatory protein gene (lrp), methylglyoxal synthase gene (mgsA), phosphogluconate dehydratase gene (edd), phosphoglycerate dehydrogenase gene (serA), phosphoglycerate kinase gene (pgk), ribulose-phosphate 3-epimerase gene (rpe), RNA polymerase-binding transcription factor gene (dksA), serine hydroxymethyltransferase gene (glyA), transaldolase A gene (talA), transaldolase B gene (talB), or a combination thereof.

### BRIEF DESCRIPTION OF THE DRAWINGS

FIG. 1 illustrates native upper central carbon metabolism in Escherichia coli expressing heterologous methanol assimilation pathway genes, including methanol dehydrogenase gene (mdh), hexulose phosphate synthase gene (hps), and hexulose phosphate isomerase gene (phi). The glucose-6-phosphate isomerase (pgi), phosphogluconate dehydratase (edd), 2-dehydro-3-deoxy-D-gluconate 6-phosphate (KDPG) aldolase (eda), 2,3-bisphosphoglycerate-dependent phosphoglycerate mutase (gpmA), 2,3-bisphosphoglycerate-independent phosphoglycerate mutase (gpmM), glyceraldehyde-3-phosphate dehydrogenase (gapA), glyceraldehyde-3-phosphate dehydrogenase (gapC), phosphoglycerate kinase (pgk), enolase (eno), fructose-1,6-bisphosphatase class 1 (fbp), fructose-1,6-bisphosphatase 1 class 2 (glpX), 6-phosphogluconate dehydrogenase (gnd), transaldolase A (talA), transaldolase B (talB), phosphofructokinase (pfk), fructose bisphosphate aldolase (fba), transketolase (tkt), ribose-5-phosphate isomerase (rpi), ribulose phosphate epimerase (rpe), and S-(hydroxymethyl)glutathione dehydrogenase (frmA) genes are indicated next to the respective reactions they catalyze.

FIG. 2 shows growth dynamics of recombinant Escherichia coli expressing Bacillus stearothermophilus 2334 (Bst) methanol dehydrogenase (MDH), Bacillus methanolicus MGA3 (Bme) hexulose phosphate synthase (HPS) and Bacillus methanolicus MGA3 (Bme) hexulose phosphate isomerase (PHI). The recombinant Escherichia coli cells were modified to contain deletions of the native formaldehyde dehydrogenase (frmA) and glucose-6-phosphate isomerase (pgi) genes. Cells were grown in minimal media containing 1 gram per liter of yeast extract and supplemented with methanol as a co-substrate. As illustrated, cultures supplemented with methanol demonstrate higher biomass yields compared with those not supplemented with methanol. This indicates that recombinant Escherichia coli cells containing deletions of the native formaldehyde dehydrogenase (frmA) and glucose-6-phosphate isomerase (pgi) genes can oxidize methanol, which leads to methanol assimilation in a synthetic methylotrophic organism.

FIG. 3 shows growth dynamics of recombinant Escherichia coli expressing Bacillus stearothermophilus 2334 (Bst) methanol dehydrogenase (MDH), Bacillus methanolicus MGA3 (Bme) hexulose phosphate synthase (HPS) and Bacillus methanolicus MGA3 (Bme) hexulose phosphate isomerase (PHI). The recombinant Escherichia coli cells were modified to contain deletions of the native formaldehyde dehydrogenase (frmA), glucose-6-phosphate isomerase (pgi) and phosphogluconate dehydratase (edd) genes. Cells were grown in minimal media containing 1 gram per liter of glucose and supplemented with methanol as a co-substrate. As illustrated, cultures supplemented with methanol demonstrate higher biomass yields compared with those not supplemented with methanol. This indicates that recombinant Escherichia coli cells containing deletions of the native formaldehyde dehydrogenase (frmA), glucose-6-phosphate isomerase (pgi) and phosphogluconate dehydratase (edd) genes can oxidize methanol, which leads to methanol assimilation in a synthetic methylotrophic organism.

FIG. 4 shows the average carbon labeling of intracellular metabolites and amino acids in recombinant Escherichia coli expressing Bacillus stearothermophilus 2334 (Bst) methanol dehydrogenase (MDH), Bacillus methanolicus MGA3 (Bme) hexulose phosphate synthase (HPS) and Bacillus methanolicus MGA3 (Bme) hexulose phosphate isomerase (PHI). The recombinant Escherichia coli cells were modified to contain deletions of the native formaldehyde dehydrogenase (frmA) and glucose-6-phosphate isomerase (pgi) genes. Cells were grown in minimal media containing 0.5 gram per liter of glucose and supplemented with ¹³C-methanol as a co-substrate. As illustrated, cells containing a deletion of pgi demonstrate higher conversion of methanol to metabolites compared with cells containing intact pgi. This indicates that recombinant Escherichia coli cells containing deletions of the native formaldehyde dehydrogenase (frmA) and glucose-6-phosphate isomerase (pgi) genes can oxidize methanol, which leads to methanol assimilation in a synthetic methylotrophic organism. Intracellular metabolites and amino acids: glycine (Gly), serine (Ser), pyruvate (Pyr), alanine (Ala), 3-phosphoglycerate (3PG), valine (Val), glutamine (Gin), isoleucine (Ile), methionine (Met), malate (Mal), threonine (Thr).

FIG. 5 illustrates heterologous aerobic biosynthetic pathways for acetone, isopropanol, 1,3-butanediol and 1-butanol production in Escherichia coli. All metabolites and chemicals are derived from acetyl-CoA as indicated. Acetone pathway enzymes comprise heterologous acetoacetate decarboxylase (ADC), acetoacetate CoA-transferases (CTFA and/or CTFB), and thiolase (THL).

FIG. 6 shows acetone production profiles of recombinant Escherichia coli expressing Bacillus stearothermophilus 2334 (Bst) methanol dehydrogenase (MDH), Bacillus methanolicus MGA3 (Bme) hexulose phosphate synthase (HPS), Bacillus methanolicus MGA3 (Bme) hexulose phosphate isomerase (PHI), Clostridium acetobutylicum (Cac) acetoacetate decarboxylase (ADC), Clostridium acetobutylicum (Cac) coenzyme A transferase (CTFAB) and Clostridium acetobutylicum (Cac) thiolase (THL). The recombinant Escherichia coli cells were modified to contain deletions of the native formaldehyde dehydrogenase (frmA) and glucose-6-phosphate isomerase (pgi) genes. Cells were grown in minimal media containing 10 grams per liter of yeast extract and supplemented with glucose and methanol as a co-substrate. As illustrated, cultures supplemented with methanol demonstrate higher acetone titers compared with those not supplemented with methanol. This indicates that recombinant Escherichia coli cells containing deletions of the native formaldehyde dehydrogenase (frmA) and glucose-6-phosphate isomerase (pgi) genes can oxidize methanol in the presence of glucose, which leads to enhanced methanol assimilation that improves acetone titers in a synthetic methylotrophic organism.

FIG. 7 shows methanol consumption profiles of recombinant Escherichia coli expressing Bacillus stearothermophilus 2334 (Bst) methanol dehydrogenase (MDH), Bacillus methanolicus MGA3 (Bme) hexulose phosphate synthase (HPS), Bacillus methanolicus MGA3 (Bme) hexulose phosphate isomerase (PHI), Clostridium acetobutylicum (Cac) acetoacetate decarboxylase (ADC), Clostridium acetobutylicum (Cac) coenzyme A transferase (CTFAB) and Clostridium acetobutylicum (Cac) thiolase (THL). The recombinant Escherichia coli cells were modified to contain deletions of the native formaldehyde dehydrogenase (frmA) and glucose-6-phosphate isomerase (pgi) genes. Cells were grown in minimal media containing 10 grams per liter of yeast extract and supplemented with glucose and methanol as a co-substrate. As illustrated, cells containing a deletion of pgi demonstrate higher methanol consumption compared with cells containing intact pgi. This indicates that recombinant Escherichia coli cells containing deletions of the native formaldehyde dehydrogenase (frmA) and glucose-6-phosphate isomerase (pgi) genes can oxidize methanol in the presence of glucose, which leads to enhanced methanol assimilation in a synthetic methylotrophic organism.

FIG. 8 shows the relative abundance of the M+1 acetone mass isotopomer at 48 hours in recombinant Escherichia coli expressing Bacillus stearothermophilus 2334 (Bst) methanol dehydrogenase (MDH), Bacillus methanolicus MGA3 (Bme) hexulose phosphate synthase (HPS), Bacillus methanolicus MGA3 (Bme) hexulose phosphate isomerase (PHI), Clostridium acetobutylicum (Cac) acetoacetate decarboxylase (ADC), Clostridium acetobutylicum (Cac) coenzyme A transferase (CTFAB) and Clostridium acetobutylicum (Cac) thiolase (THL). The recombinant Escherichia coli cells were modified to contain deletions of the native formaldehyde dehydrogenase (frmA) and glucose-6-phosphate isomerase (pgi) genes. Cells were grown in minimal media containing 10 grams per liter of yeast extract and supplemented with glucose and ¹³C-methanol as a co-substrate. As illustrated, cells containing a deletion of pgi demonstrate higher conversion of methanol carbon to acetone compared with cells containing intact pgi. This indicates that recombinant Escherichia coli cells containing deletions of the native formaldehyde dehydrogenase (frmA) and glucose-6-phosphate isomerase (pgi) genes can oxidize methanol in the presence of glucose, which leads to enhanced methanol assimilation in a synthetic methylotrophic organism. The labeling profile was not corrected for natural ¹³C abundance here.

FIG. 9 shows the relative abundance of the M+1 n-butanol mass isotopomer at 48 hours in recombinant Escherichia coli expressing Bacillus stearothermophilus 2334 (Bst) methanol dehydrogenase (MDH), Bacillus methanolicus MGA3 (Bme) hexulose phosphate synthase (HPS), Bacillus methanolicus MGA3 (Bme) hexulose phosphate isomerase (PHI), phaA (acetyltransferase from Ralstonia eutropha), phaB (NADPH-dependent acetoacetyl-CoA reductase from R. eutropha), bld (butylraldehyde dehydrogenase from Clostridium saccharoperbutylacetonicum), phaJ ((R)-specific enoyl-CoA hydratase from Aeromonas caviae), and ter (trans-enoyl-CoA reductase from Treponema denticola). The recombinant Escherichia coli cells were modified to contain deletions of the native formaldehyde dehydrogenase (frmA) and glucose-6-phosphate isomerase (pgi) genes. Cells were grown in minimal media containing 10 grams per liter of yeast extract and supplemented with glucose and ¹³C-methanol as a co-substrate. As illustrated, cells containing a deletion of pgi demonstrate higher conversion of methanol carbon to n-butanol compared with cells containing intact pgi. This indicates that recombinant Escherichia coli cells containing deletions of the native formaldehyde dehydrogenase (frmA) and glucose-6-phosphate isomerase (pgi) genes can oxidize methanol in the presence of glucose, which leads to enhanced methanol assimilation in a synthetic methylotrophic organism. The labeling profile was corrected for natural ¹³C abundance here.

FIG. 10 shows growth dynamics of recombinant Escherichia coli expressing Bacillus stearothermophilus 2334 (Bst) methanol dehydrogenase (MDH), Bacillus methanolicus MGA3 (Bme) hexulose phosphate synthase (HPS), Bacillus methanolicus MGA3 (Bme) hexulose phosphate isomerase (PHI), Bacillus methanolicus MGA3 (Bme) phosphofructokinase (PFK), Bacillus methanolicus MGA3 (Bme) fructose bisphosphate aldolase (FBA), Bacillus methanolicus MGA3 (Bme) transketolase (TKT), Bacillus methanolicus MGA3 (Bme) fructose/sedoheptulose biphosphatase (GLPX), and Bacillus methanolicus MGA3 (Bme) ribulose phosphate epimerase (RPE). The recombinant Escherichia coli cells were modified to contain deletions of the native formaldehyde dehydrogenase (frmA) gene. Cells were grown in minimal media containing 1 gram per liter of yeast extract and supplemented with methanol as a co-substrate. As illustrated, cultures supplemented with methanol demonstrate higher biomass yields compared with those not supplemented with methanol. This indicates that recombinant Escherichia coli cells containing deletions of the native formaldehyde dehydrogenase (frmA) gene can oxidize methanol, which leads to methanol assimilation in a synthetic methylotrophic organism.

FIG. 11 shows the average carbon labeling of intracellular metabolites and amino acids in recombinant Escherichia coli expressing Bacillus stearothermophilus 2334 (Bst) methanol dehydrogenase (MDH), Bacillus methanolicus MGA3 (Bme) hexulose phosphate synthase (HPS) and Bacillus methanolicus MGA3 (Bme) hexulose phosphate isomerase (PHI), Bacillus methanolicus MGA3 (Bme) phosphofructokinase (PFK), Bacillus methanolicus MGA3 (Bme) fructose bisphosphate aldolase (FBA), Bacillus methanolicus MGA3 (Bme) transketolase (TKT), Bacillus methanolicus MGA3 (Bme) fructose/sedoheptulose biphosphatase (GLPX), and Bacillus methanolicus MGA3 (Bme) ribulose phosphate epimerase (RPE). The recombinant Escherichia coli cells were modified to contain deletions of the native formaldehyde dehydrogenase (frmA) gene. Cells were grown in minimal media containing 0.5 gram per liter of yeast extract and supplemented with ¹³C-methanol as a co-substrate. As illustrated, cells expressing heterologous PPP genes demonstrate higher conversion of methanol to metabolites compared with cells not expressing heterologous PPP genes. This indicates that recombinant Escherichia coli cells containing deletions of the native formaldehyde dehydrogenase (frmA) gene can oxidize methanol, which leads to methanol assimilation in a synthetic methylotrophic organism. Intracellular metabolites and amino acids: glycine (Gly), glutamate (Glu), serine (Ser), glycogen (Glyc), succinate (Suc), malate (Mal), alanine (Ala), citrate (Cit), pyruvate (Pyr), 3-phosphoglycerate (3PG), phosphoenolpyruvate (PEP).

FIG. 12 illustrates the regulatory network of the leucine-responsive regulatory protein (lrp) in Escherichia coli. Pathway upregulation is indicated by (+) while pathway downregulation is indicated by (−).

FIG. 13 shows growth dynamics of recombinant Escherichia coli expressing Bacillus stearothermophilus 2334 (Bst) methanol dehydrogenase (MDH), Bacillus methanolicus MGA3 (Bme) hexulose phosphate synthase (HPS) and Bacillus methanolicus MGA3 (Bme) hexulose phosphate isomerase (PHI). The recombinant Escherichia coli cells were modified to contain deletions of the native formaldehyde dehydrogenase (frmA) and leucine-responsive regulatory protein (lrp) genes. Cells were grown in minimal media containing 1 gram per liter of yeast extract and supplemented with methanol as a co-substrate. As illustrated, cultures supplemented with methanol demonstrate higher biomass yields compared with those not supplemented with methanol. This indicates that recombinant Escherichia coli cells containing deletions of the native formaldehyde dehydrogenase (frmA) and leucine-responsive regulatory protein (lrp) genes can oxidize methanol, which leads to methanol assimilation in a synthetic methylotrophic organism.

FIG. 14 shows the relative abundance profile of glycogen and RNA ¹³C labeling for recombinant Escherichia coli expressing Bacillus stearothermophilus 2334 (Bst) methanol dehydrogenase (MDH), Bacillus methanolicus MGA3 (Bme) hexulose phosphate synthase (HPS) and Bacillus methanolicus MGA3 (Bme) hexulose phosphate isomerase (PHI). The recombinant Escherichia coli cells were modified to contain a deletion of the native formaldehyde dehydrogenase (frmA) gene, indicated as the ‘Base’ strain, or deletions of the native formaldehyde dehydrogenase (frmA) and leucine-responsive regulatory protein (lrp) genes, indicated as the ‘Δlrp’ strain. Cells were grown in minimal media containing 1 gram per liter of yeast extract and supplemented with ¹³C-methanol as a co-substrate. As illustrated, the ‘Δlrp’ strain demonstrates higher labeling in both glycogen and RNA compared to the ‘Base’ strain. This indicates that the ‘Δlrp’ strain enhances methanol assimilation in a synthetic methylotrophic organism.

FIG. 15 shows the absolute level and ¹³C labeled fraction of glycogen and RNA for recombinant Escherichia coli expressing Bacillus stearothermophilus 2334 (Bst) methanol dehydrogenase (MDH), Bacillus methanolicus MGA3 (Bme) hexulose phosphate synthase (HPS) and Bacillus methanolicus MGA3 (Bme) hexulose phosphate isomerase (PHI). The recombinant Escherichia coli cells were modified to contain a deletion of the native formaldehyde dehydrogenase (frmA) gene, indicated as the ‘Base’ strain, or deletions of the native formaldehyde dehydrogenase (frmA) and leucine-responsive regulatory protein (lrp) genes, indicated as the ‘Δlrp’ strain. Cells were grown in minimal media containing 1 gram per liter of yeast extract and supplemented with ¹³C-methanol as a co-substrate. As illustrated, the ‘Δlrp’ strain demonstrates higher ¹³C labeling and lower degradation in both glycogen and RNA compared to the ‘Base’ strain. This indicates that the ‘Δlrp’ strain enhances methanol assimilation in a synthetic methylotrophic organism.

FIG. 16 shows growth dynamics of recombinant Escherichia coli expressing Bacillus stearothermophilus 2334 (Bst) methanol dehydrogenase (MDH), Bacillus methanolicus MGA3 (Bme) hexulose phosphate synthase (HPS) and Bacillus methanolicus MGA3 (Bme) hexulose phosphate isomerase (PHI). The recombinant Escherichia coli cells were modified to contain deletions of the native formaldehyde dehydrogenase (frmA) and GDP pyrophosphokinase/GTP pyrophosphokinase (relA) genes. Cells were grown in minimal media containing 1 gram per liter of yeast extract and supplemented with methanol as a co-substrate. As illustrated, cultures supplemented with methanol demonstrate growth and higher biomass yields compared with those not supplemented with methanol, which do not yield cell growth. This indicates that recombinant Escherichia coli cells containing deletions of the native formaldehyde dehydrogenase (frmA) and GDP pyrophosphokinase/GTP pyrophosphokinase (relA) genes can oxidize methanol, which leads to methanol assimilation in a synthetic methylotrophic organism.

FIG. 17 shows growth dynamics of recombinant Escherichia coli expressing Bacillus stearothermophilus 2334 (Bst) methanol dehydrogenase (MDH), Bacillus methanolicus MGA3 (Bme) hexulose phosphate synthase (HPS) and Bacillus methanolicus MGA3 (Bme) hexulose phosphate isomerase (PHI). The recombinant Escherichia coli cells were modified to contain deletions of the native formaldehyde dehydrogenase (frmA) and RNA polymerase-binding transcription factor (dksA) genes. Cells were grown in minimal media containing 1 gram per liter of yeast extract and supplemented with methanol as a co-substrate. As illustrated, cultures supplemented with methanol demonstrate growth and higher biomass yields compared with those not supplemented with methanol, which do not yield cell growth. This indicates that recombinant Escherichia coli cells containing deletions of the native formaldehyde dehydrogenase (frmA) and RNA polymerase-binding transcription factor (dksA) genes can oxidize methanol, which leads to methanol assimilation in a synthetic methylotrophic organism.

FIG. 18 shows growth dynamics of recombinant Escherichia coli expressing Bacillus stearothermophilus 2334 (Bst) methanol dehydrogenase (MDH), Bacillus methanolicus MGA3 (Bme) hexulose phosphate synthase (HPS) and Bacillus methanolicus MGA3 (Bme) hexulose phosphate isomerase (PHI). The recombinant Escherichia coli cells were modified to contain deletions of the native formaldehyde dehydrogenase (frmA) and ribulose-phosphate 3-epimerase (rpe) genes. Cells were grown in minimal media containing 1 gram per liter of ribose and supplemented with methanol as a co-substrate. As illustrated, cultures supplemented with methanol demonstrate growth and higher biomass yields compared with those not supplemented with methanol, which do not yield cell growth. This indicates that recombinant Escherichia coli cells containing deletions of the native formaldehyde dehydrogenase (frmA) and ribulose-phosphate 3-epimerase (rpe) genes can oxidize methanol, which leads to methanol assimilation in a synthetic methylotrophic organism.

FIG. 19 shows growth dynamics of recombinant Escherichia coli expressing Bacillus stearothermophilus 2334 (Bst) methanol dehydrogenase (MDH), Bacillus methanolicus MGA3 (Bme) hexulose phosphate synthase (HPS) and Bacillus methanolicus MGA3 (Bme) hexulose phosphate isomerase (PHI). The recombinant Escherichia coli cells were modified to contain deletions of the native formaldehyde dehydrogenase (frmA), ribulose-phosphate 3-epimerase (rpe), and phosphogluconate dehydratase (edd) genes. Cells were grown in minimal media containing 1 gram per liter of gluconate and supplemented with methanol as a co-substrate. As illustrated, cultures supplemented with methanol demonstrate growth and higher biomass yields compared with those not supplemented with methanol, which do not yield cell growth. This indicates that recombinant Escherichia coli cells containing deletions of the native formaldehyde dehydrogenase (frmA), ribulose-phosphate 3-epimerase (rpe), and phosphogluconate dehydratase (edd) genes can oxidize methanol, which leads to methanol assimilation in a synthetic methylotrophic organism.

FIG. 20 shows growth dynamics of recombinant Escherichia coli expressing Bacillus stearothermophilus 2334 (Bst) methanol dehydrogenase (MDH), Bacillus methanolicus MGA3 (Bme) hexulose phosphate synthase (HPS) and Bacillus methanolicus MGA3 (Bme) hexulose phosphate isomerase (PHI). The recombinant Escherichia coli cells were modified to contain deletions of the native phosphoglycerate dehydrogenase (serA), aminomethyltransferase (gcvT), glycine cleavage system H protein (gcvH), and glycine decarboxylase (gcvP) genes. Cells were grown in minimal media containing glucose and supplemented with methanol as a co-substrate. As illustrated, cultures supplemented with methanol demonstrate growth and higher biomass yields compared with those not supplemented with methanol, which do not yield cell growth. This indicates that recombinant Escherichia coli cells containing deletions of the native phosphoglycerate dehydrogenase (serA), aminomethyltransferase (gcvT), glycine cleavage system H protein (gcvH), and glycine decarboxylase (gcvP) genes can oxidize methanol, which leads to methanol assimilation in a synthetic methylotrophic organism.

FIG. 21 shows the growth profile of recombinant Escherichia coli expressing Bacillus stearothermophilus 2334 (Bst) methanol dehydrogenase (MDH), Bacillus methanolicus MGA3 (Bme) hexulose phosphate synthase (HPS) and Bacillus methanolicus MGA3 (Bme) hexulose phosphate isomerase (PHI). The recombinant Escherichia coli cells were modified to contain a deletion of the native formaldehyde dehydrogenase (frmA) gene. Cells were grown in minimal media containing 1 gram per liter of each growth substrate and supplemented with methanol as a co-substrate. As illustrated, methanol supplementation improves biomass yield for a variety of growth substrates, including single amino acids. This indicates that a variety of growth substrates, including single amino acids, enhances methanol assimilation in a synthetic methylotrophic organism.

### DETAILED DESCRIPTION OF THE INVENTION

The present invention relates to further engineering non-naturally occurring (i.e., recombinant) methylotrophic microbes (i.e., microorganisms) such as Escherichia coli (E. coli) for increasing production of one or more metabolites. The non-naturally occurring methylotrophic microbes are capable of using methanol for growth, for example, as co-substrate together with various carbohydrates or other carbon and energy substrates and producing metabolites, but derived from microbes that do not naturally grow on or metabolize methanol. The resulting non-naturally occurring (i.e., recombinant or synthetic) microbes are capable of using the reduction energy from methanol utilization to produce liquid fuel and chemicals. This technology integrates all critical components required for achieving the overall goal of cost-efficient biofuel production starting from methanol (and ultimately CH₄) while at the same time minimizing release of greenhouse gases, such as CO₂, which cause climate change.

The present invention provides a method for increasing production of a metabolite by a non-naturally occurring methylotroph. The method comprises growing the non-naturally occurring methylotroph in a medium comprising methanol. Expression of one or more native genes is changed in the non-naturally occurring methylotroph.

The method may further comprise increasing production of the metabolite by the non-naturally occurring methylotroph by at least about 5%, 10%, 15%, 20%, 30%, 40%, 50%, 60%, 70%, 80%, 90%, 95%, 99%, 100% or 200% as compared with that by a control microbe, which is identical to the non-naturally occurring methylotroph except that the expression of the one or more native genes are not changed.

Also provided is a corresponding non-naturally occurring methylotroph, in which expression of the one or more native genes is changed (e.g., increased or decreased) by, for example, at least about 5%, 10%, 15%, 20%, 30%, 40%, 50%, 60%, 70%, 80%, 90%, 95%, 99% or 100%.

The term “microbe” used herein refers to a single cell organism. Examples of microbes include bacteria, archaea, and fungi.

The terms “methylotroph,” “methylotrophic microorganism” and “methylotrophic microbe” are used herein interchangeably and refer to a microbe capable of metabolizing a one-carbon compound, such as methane or methanol, into its cell mass, a metabolite or a combination thereof.

The terms “non-methylotroph,” “non-methylotrophic microorganism” and “non-methylotrophic microbe” are used herein interchangeably and refer to a microbe incapable of metabolizing a one-carbon compound, such as methane or methanol, into its cell mass, a metabolite or a combination thereof.

The terms “non-naturally occurring methylotroph,” “non-naturally occurring methylotrophic microorganism” and “methylotrophic microbe” are used herein interchangeably and refer to a methylotroph that has been prepared by modifying one or more native genes and/or expressing one or more heterologous genes in a non-methylotroph.

The non-naturally occurring methylotroph is a microbe selected from the group consisting of facultative aerobic organisms, facultative anaerobic organisms and anaerobic organisms. The non-naturally occurring methylotroph may be a microbe derived from a microbe selected from the group consisting of phyla Proteobacteria, Firmicutes, Actinobacteria, Cyanobacteria, Chlorobi and Deinococcus-Thermus. In some embodiments, the non-naturally occurring methylotroph is a microbe derived from a microbe selected from the group consisting of Acetobacter, Acinetobacter, Bacillus, Chlorobi, Clostridium, Corynebacterium, Cyanobacteria, Deinococcus, Enterobacter, Enterobacteria, Escherichia, Geobacillus, Geobacter, Klebsiella, Lactobacillus, Lactococcus, Mannheimia, Propionibacterium, Pseudomonas, Ralstonia, Shewanella, Streptococcus, Streptomyces, Synechococcus, Synechocystis and Zymomonas. In one embodiment, the non-naturally occurring methylotroph is Escherichia coli. The non-naturally occurring methylotroph may be a microbe comprising a deletion of a formaldehyde-dissimilation pathway, such as an frmRAB operon.

The non-naturally occurring methylotrophs may be prepared by any techniques known in the art. Some non-naturally occurring methylotrophs are described in WO 2015/108777 A1. The non-naturally occurring methylotroph may express one or more heterologous genes. For example, the non-naturally occurring methylotroph may express a heterologous enzyme capable of converting methanol to formaldehyde (HCHO) such as heterologous methanol dehydrogenase (MDH). The expression of the heterologous MDH may be under control of a formaldehyde responsive promoter.

The non-naturally occurring methylotroph may further express one or more heterologous ribulose monophosphate (RuMP) pathway enzymes such as 3-hexulose-6-phosphate synthase (HPS) and 3-hexulose-6-phosphate isomerase (PHI). The expression of heterologous HPS and PHI may be under control of a formaldehyde-responsive promoter.

The non-naturally occurring methylotroph may further express one or more heterologous pentose-phosphate pathway (PPP) enzymes such as phosphofructokinase (PFK), fructose bisphosphate aldolase (FBA), transketolase (TKT), fructose/sedoheptulose bisphosphatase (GLPX), ribulose phosphate epimerase (RPE), ribose-5-phosphate isomerase (RPI) and transaldolase (TAL). The expression of the heterologous PPP enzymes (e.g., PFK, FBA, TKT, GLPX, RPE, RPI, and TAL) may be under control of a formaldehyde-responsive promoter. In one embodiment, the non-naturally occurring methylotroph expresses heterologous MDH, heterologous HPS, and heterologous PHI. In another embodiment, the non-naturally occurring methylotroph expresses heterologous MDH, heterologous HPS, heterologous PHI, heterologous PFK, heterologous FBA, heterologous TKT, heterologous GLPX, heterologous RPE, heterologous RPI and heterologous TAL. In yet another embodiment, the non-naturally occurring methylotroph expresses heterologous MDH, heterologous HPS, heterologous PHI, heterologous PFK, heterologous FBA, heterologous TKT, heterologous GLPX and heterologous RPE.

The non-naturally occurring methylotroph may further express heterologous CO₂ fixation pathway enzymes such as carbonic anhydrase (CA, EC 4.2.1.1), formate dehydrogenase (FDH, EC 1.2.1.43 or EC1.2.1.2), formaldehyde dehydrogenase (FLD, EC 1.1.1.284); heterologous enzymes of the reductive tricarboxylic acid cycle such as ATP citrate lyase (ACL), 2-oxoglutarate: ferredoxin oxidoreductase (OGOR), isocitrate dehydrogenase (ICDH), and fumarate reductase (FR); heterologous enzymes of the glycine cleavage system such as aminomethyltransferase (AMT), dehydrolipoyl dehydrogenase (LPDH), glycine dehydrogenase (GDH); and heterologous enzymes of the non-oxidative glycolysis pathway such as fructose phosphoketolase, xylose phosphoketolase, transaldolase, transketolase, fructose 1,2-bisphosphate aldolase, fructose 1,6-bisphosphatase, ribulose-5-phosphate epimerase, ribose-5-phosphate isomerase, and trios phosphate isomerase. In one embodiment, the non-naturally occurring methylotroph expresses heterologous MDH, heterologous HPS, heterologous PHI, heterologous PFK, heterologous FBA, heterologous TKT, heterologous GLPX, heterologous RPE, heterologous RPI, heterologous TAL, heterologous PGI, heterologous ZWF, heterologous PGL, heterologous GND, heterologous CA, heterologous FDH, and heterologous FLD.

The non-naturally occurring microbe may further express heterologous dihydroxyacetone synthase (DHAS, EC=2.2.1.3), which is also known as formaldehyde transketolase or glycerone synthase. Additionally, the non-naturally occurring methylotroph may further express heterologous dihydroxyacetone kinase (DAK, EC=2.7.1.29), which is also known as glycerone kinase. In one embodiment, the non-naturally occurring methylotroph expresses heterologous MDH, heterologous HPS, heterologous PHI, heterologous PFK, heterologous FBA, heterologous TKT, heterologous GLPX, heterologous TAL, heterologous RPI, heterologous RPE, heterologous PGI, heterologous ZWF, heterologous PGL, heterologous GND heterologous CA, heterologous FDH, heterologous FLD, heterologous DHAS, and heterologous DAK.

In the non-naturally occurring methylotroph, expression of native genes may be changed individually or in a group as described below. The native genes may comprise one or more deletions. The native genes may be deleted from the genome of the non-naturally occurring methylotroph. The native genes may be inactivated. For example, the expression of the native genes in the non-naturally occurring methylotroph may be changed (e.g., increased or decreased) by at least about 5%, 10%, 15%, 20%, 30%, 40%, 50%, 60%, 70%, 80%, 90%, 95%, 99% or 100%. Examples of the native genes whose expression is changed include 2,3-bisphosphoglycerate-dependent phosphoglycerate mutase gene (gpmA), 2,3-bisphosphoglycerate-independent phosphoglycerate mutase gene (gpmM), 2-dehydro-3-deoxy-D-gluconate 6-phosphate (KDPG) aldolase gene (eda), 6-phosphogluconate dehydrogenase gene (gnd), aminomethyltransferase gene (gcvT), bifunctional (p)ppGpp synthetase II/guanosine-3′,5′-bis pyrophosphate 3′-pyrophosphohydrolase gene (spoT), enolase gene (eno), fructose-1,6-bisphosphatase 1 class 2 gene (glpX), fructose-1,6-bisphosphatase class 1 gene (fbp), GDP pyrophosphokinase/GTP pyrophosphokinase gene (relA), glucose-6-phosphate isomerase gene (pgi), glyceraldehyde-3-phosphate dehydrogenase gene (gapA), glyceraldehyde-3-phosphate dehydrogenase gene (gapC), glycine cleavage system H protein gene (gcvH), glycine decarboxylase gene (gcvP), HTH-type transcriptional regulator GntR gene (gntR), leucine-responsive regulatory protein gene (lrp), methylglyoxal synthase gene (mgsA), phosphogluconate dehydratase gene (edd), phosphoglycerate dehydrogenase gene (serA), phosphoglycerate kinase gene (pgk), ribulose-phosphate 3-epimerase gene (rpe), RNA polymerase-binding transcription factor gene (dksA), serine hydroxymethyltransferase gene (glyA), transaldolase A gene (talA), transaldolase B gene (talB), or a combination thereof.

Also provided is a corresponding non-naturally occurring methylotroph, in which expression of one or more of these native genes (i.e., 2,3-bisphosphoglycerate-dependent phosphoglycerate mutase gene (gpmA), 2,3-bisphosphoglycerate-independent phosphoglycerate mutase gene (gpmM), 2-dehydro-3-deoxy-D-gluconate 6-phosphate (KDPG) aldolase gene (eda), 6-phosphogluconate dehydrogenase gene (gnd), aminomethyltransferase gene (gcvT), bifunctional (p)ppGpp synthetase II/guanosine-3′,5′-bis pyrophosphate 3′-pyrophosphohydrolase gene (spoT), enolase gene (eno), fructose-1,6-bisphosphatase 1 class 2 gene (glpX), fructose-1,6-bisphosphatase class 1 gene (fbp), GDP pyrophosphokinase/GTP pyrophosphokinase gene (relA), glucose-6-phosphate isomerase gene (pgi), glyceraldehyde-3-phosphate dehydrogenase gene (gapA), glyceraldehyde-3-phosphate dehydrogenase gene (gapC), glycine cleavage system H protein gene (gcvH), glycine decarboxylase gene (gcvP), HTH-type transcriptional regulator GntR gene (gntR), leucine-responsive regulatory protein gene (lrp), methylglyoxal synthase gene (mgsA), phosphogluconate dehydratase gene (edd), phosphoglycerate dehydrogenase gene (serA), phosphoglycerate kinase gene (pgk), ribulose-phosphate 3-epimerase gene (rpe), RNA polymerase-binding transcription factor gene (dksA), serine hydroxymethyltransferase gene (glyA), transaldolase A gene (talA), and/or transaldolase B gene (talB)) is changed, for example, increased or decreased by at least about 5%, 10%, 15%, 20%, 30%, 40%, 50%, 60%, 70%, 80%, 90%, 95%, 99% or 100%. This non-naturally occurring methylotroph may be prepared by changing expression of the one or more native genes in a non-naturally occurring methylotroph, for example, deleting the one or more native genes from the genome of a non-naturally occurring methylotroph. In some embodiments, the non-naturally occurring methylotroph expresses heterologous methanol dehydrogenase. In other embodiments, the non-naturally occurring methylotroph expresses heterologous 3-hexulose-6-phosphate synthase (HPS) and heterologous 3-hexulose-6-phosphate isomerase (PHI). In yet other embodiments, the expression of the heterologous HPS and the heterologous PHI in the non-naturally occurring methylotroph are under control of a formaldehyde-responsive promoter.

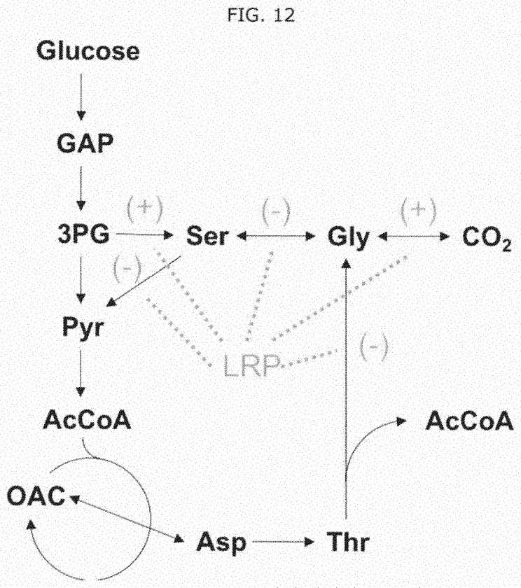

The leucine-responsive regulatory protein (Lrp) regulates metabolism of threonine to glycine to serine (FIG. 12) by down-regulating expression of genes involved in threonine catabolism encoding L-threonine 3-dehydrogenase (tdh), 2-amino-3-ketobutyrate CoA ligase (kbl) and serine hydroxymethyltranserase (glyA), which catalyze conversion of threonine to L-2-amino-3-oxobutanoate, L-2-amino-3-oxobutanoate to glycine, and glycine to serine, respectively. Deletion of lrp eliminates this down-regulation and thus allows increased expression of tdh, kbl, and glyA. In one embodiment, the method comprises growing a non-naturally occurring methylotroph in a medium comprising methanol, and native leucine-responsive regulatory protein gene (lrp) is deleted from the genome of the non-naturally occurring methylotroph. As a result, expression of native tdh, kbl, glyA or a combination thereof is upregulated.

Other native genes regulated by LRP in the non-naturally occurring methylotroph may include ilvI (acetolactate synthase/acetohydroxybutanoate synthase, catalytic subunit), ilvH (acetolactate synthase/acetohydroxybutanoate synthase, regulatory subunit), serC (phosphoserine/phosphohydroxythreonine aminotransferase), aroA (3-phosphoshikimate 1-carboxyvinyltransferase), dadA (D-amino acid dehydrogenase), dadX (alanine racemase 2), adhE (aldehyde-alcohol dehydrogenase), lrp (DNA-binding transcriptional dual regulator Lrp), oppA (oligopeptide ABC transporter periplasmic binding protein), oppB (murein tripeptide ABC transporter/oligopeptide ABC transporter inner membrane subunit OppB), oppC (murein tripeptide ABC transporter/oligopeptide ABC transporter inner membrane subunit OppC), oppD (murein tripeptide ABC transporter/oligopeptide ABC transporter ATP binding subunit OppD), oppF (murein tripeptide ABC transporter/oligopeptide ABC transporter ATP binding subunit OppF), osmC (osmotically inducible peroxiredoxin), sdaA (L-serine deaminase I), lysP (lysine:H⁺ symporter), ompC (outer membrane porin C), micF (small regulatory RNA MicF), stpA (H-NS-like DNA-binding transcriptional repressor with RNA chaperone activity), gcvT (aminomethyltransferase), gcvH (glycine cleavage system H protein), gcvP (glycine decarboxylase), ssrS (6S RNA), fau (putative 5-formyltetrahydrofolate cyclo-ligase), serA (phosphoglycerate dehydrogenase), gltB (glutamate synthase subunit GltB), gltD (glutamate synthase subunit GltD), gltF (periplasmic protein GltF), malt (DNA-binding transcriptional activator MalT), livK (L-leucine/L-phenylalanine ABC transporter periplasmic binding protein), livH (branched chain amino acid/phenylalanine ABC transporter membrane subunit LivH), livM (branched chain amino acid/phenylalanine ABC transporter membrane subunit LivM), livG (branched chain amino acid/phenylalanine ABC transporter ATP binding subunit LivG), livF (branched chain amino acid/phenylalanine ABC transporter ATP binding subunit LivF), (ilvXGMEDA operon leader peptide), ilvX (uncharacterized protein IlvX), ilvG_1 (acetolactate synthase II subunit IlvG, N-terminal fragment (pseudogene)), ilvG 2 (pseudogene), ilvM (acetolactate synthase II subunit IlvM), ilvE (branched-chain-amino-acid aminotransferase), ilvD (dihydroxy-acid dehydratase), ilvA (threonine deaminase), lysU (lysine-tRNA ligase [multifunctional]), cadB (cadaverine:lysine antiporter), cadA (lysine decarboxylase 1), aidB (isovaleryl-CoA dehydrogenase and DNA-binding transcriptional repressor), fimE (regulator for fimA), fimA (type 1 fimbriae major subunit), fimI (putative fimbrial protein FimI), fimC (type 1 fimbriae periplasmic chaperone), fimD (type I fimbriae usher protein), fimF (type 1 fimbriae minor subunit FimF), fimG (type 1 fimbriae minor subunit FimG), fimH (type 1 fimbriae D-mannose specific adhesin), osmY (periplasmic chaperone OsmY), rrsH (16S ribosomal RNA), ileV (tRNA-Ile(GAU)), alaV (tRNA-Ala(UGC)), rrlH (23S ribosomal RNA), rrfH (5S ribosomal RNA), leuE (leucine exporter), yeiL (putative DNA-binding transcriptional regulator YeiL), yojI (ABC transporter family protein/microcin J25 efflux protein), alaA (glutamate-pyruvate aminotransferase AlaA), rrsG (16S ribosomal RNA), gltW (tRNA-Glu(UUC)), rrlG (23S ribosomal RNA), rrfG (5S ribosomal RNA), csiD (PF08943 family protein CsiD), IhgO (L-2-hydroxyglutarate oxidase), gabD (NADP+-dependent succinate-semialdehyde dehydrogenase), gabT (4-aminobutyrate aminotransferase GabT), gabP (4-aminobutyrate:H⁺ symporter), alaE (L-alanine exporter), argO (L-arginine exporter), rrsD (16S ribosomal RNA), Hell (tRNA-Ile(GAU)), alaU (tRNA-Ala(UGC)), rrlD (23S ribosomal RNA), rrfD (5S ribosomal RNA), thrV (tRNA-Thr(GGU)), rrfF (5S ribosomal RNA), livJ (branched chain amino acid/phenylalanine ABC transporter periplasmic binding protein), hdeA (periplasmic acid stress chaperone), hdeB (periplasmic acid stress chaperone), yhiD (inner membrane protein YhiD), avtA (valine-pyruvate aminotransferase), rrsC (16S ribosomal RNA), gltU (tRNA-Glu(UUC)), rrlC (23S ribosomal RNA), rrfC (5S ribosomal RNA), rrsA (16S ribosomal RNA), ileT (tRNA-Ile(GAU)), alaT (tRNA-Ala(UGC)), rriA (23S ribosomal RNA), rrfA (5S ribosomal RNA), rrsB (16S ribosomal RNA), gltT (tRNA-Glu(UUC)), rrlB (23S ribosomal RNA), rrfB (5S ribosomal RNA), rrsE (16S ribosomal RNA), gltV (tRNA-Glu(UUC)), rrlE (23S ribosomal RNA), and/or rrfE (5S ribosomal RNA). In some embodiments, the method comprises growing a non-naturally occurring methylotroph in a medium comprising methanol, and native leucine-responsive regulatory protein gene (lrp) is deleted from the genome of the non-naturally occurring methylotroph such that expression of one or more of these native genes is upregulated (e.g., dadA, oppA, ompC, micF, ssrS, lysU, osmY and rrsB) or downregulated (e.g., serC, aroA, lysP, stpA, gltB, cadB, fimA and alaA).

Also provided is a corresponding non-naturally occurring methylotroph from whose genome native lrp is deleted. This non-naturally occurring methylotroph may be prepared by deleting native lrp from the genome of a non-naturally occurring methylotroph.

The medium may further comprise a co-substrate. The term “co-substrate” used herein refers to any compound other than methanol used in combination with methanol by a non-naturally occurring methylotroph for cell mass or metabolite production. Co-substrates may include, but are not limited to, monosaccharides (e.g. glucose, fructose), amino acids (e.g. alanine, threonine), or carboxylic acids (e.g. acetate). The co-substrate may comprise one or more monosaccharides selected from the group consisting of glucose, xylose, mannose, arabinose, rhamnose, ribose and a combination thereof. The co-substrate may comprise one or more amino acids selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine and a combination thereof.

In one embodiment, the method comprises growing a non-naturally occurring methylotroph in a medium comprising methanol and glucose, where native glucose-6-phosphate isomerase gene (pgi) is deleted from the genome of the non-naturally occurring methylotroph. Native phosphogluconate dehydratase gene (edd), native 2-dehydro-3-deoxy-D-gluconate 6-phosphate (KDPG) aldolase gene (eda), or a combination thereof may be optionally deleted from the genome of the non-naturally occurring methylotroph. The method may further comprise increasing production of a metabolite by the non-naturally occurring methylotroph by at least about 5%, 10%, 15%, 20%, 30%, 40%, 50%, 60%, 70%, 80%, 90%, 95%, 99%, 100% or 200% as compared with that by a control microbe, which is identical to the non-naturally occurring methylotroph except without the deletion. Also provided is a corresponding non-naturally occurring methylotroph from whose genome (a) native pgi, (b) native pgi and native edd, (c) native pgi and native eda, or (d) native pgi, native edd and native eda are deleted. This non-naturally occurring methylotroph may be prepared by deleting (a) native pgi, (b) native pgi and edd, (c) native pgi and eda, or (d) native pgi, edd and eda from the genome of a non-naturally occurring methylotroph.

In another embodiment, the method comprises growing a non-naturally occurring methylotroph in a medium comprising methanol and threonine, where (a) native leucine-responsive regulatory protein gene (lrp), (b) native GDP pyrophosphokinase/GTP pyrophosphokinase gene (relA) and bifunctional (p)ppGpp synthetase II/guanosine-3′,5′-bis pyrophosphate 3′-pyrophosphohydrolase gene (spoT), (c) native RNA polymerase-binding transcription factor gene (dksA), or (d) a combination thereof, are deleted from the genome of the non-naturally occurring methylotroph. The method may further comprise increasing production of a metabolite by the non-naturally occurring methylotroph by at least about 5%, 10%, 15%, 20%, 30%, 40%, 50%, 60%, 70%, 80%, 90%, 95%, 99%, 100% or 200% as compared with that by a control microbe, which is identical to the non-naturally occurring methylotroph except without the deletion. Also provided is a corresponding non-naturally occurring methylotroph from whose genome (a) native lrp, (b) native relA and native spoT, (c) native dksA, (d) native lrp, relA and spoT, (e) native lrp and dksA, (f) native relA, spot and dksA, or (g) native lrp, relA, spot and dksA are deleted. This non-naturally occurring methylotroph may be prepared by deleting (a) native lrp, (b) native relA and native spoT, (c) native dksA, (d) native lrp, relA and spoT, (e) native lrp and dksA, (f) native relA, spot and dksA, or (g) native lrp, relA, spot and dksA from the genome of a non-naturally occurring methylotroph.

In yet another embodiment, the method comprises growing a non-naturally occurring methylotroph in a medium comprising methanol and ribose, where native ribulose-phosphate 3-epimerase gene (rpe) is deleted from the genome of the non-naturally occurring methylotroph. The method may further comprise increasing production of a metabolite by the non-naturally occurring methylotroph by at least about 5%, 10%, 15%, 20%, 30%, 40%, 50%, 60%, 70%, 80%, 90%, 95%, 99%, 100% or 200% as compared with that by a control microbe, which is identical to the non-naturally occurring methylotroph except without the deletion. Also provided is a corresponding non-naturally occurring methylotroph from whose genome rpe is deleted. This non-naturally occurring methylotroph may be prepared by deleting rpe from the genome of a non-naturally occurring methylotroph.

In yet another embodiment, the method comprises growing a non-naturally occurring methylotroph in a medium comprising methanol and gluconate, where ribulose-phosphate 3-epimerase gene (rpe), native phosphogluconate dehydratase gene (edd), native 2-dehydro-3-deoxy-D-gluconate 6-phosphate (KDPG) aldolase gene (eda), or a combination thereof are deleted from the genome of the non-naturally occurring methylotroph. The method may further comprise increasing production of a metabolite by the non-naturally occurring methylotroph by at least about 5%, 10%, 15%, 20%, 30%, 40%, 50%, 60%, 70%, 80%, 90%, 95%, 99%, 100% or 200% as compared with that by a control microbe, which is identical to the non-naturally occurring methylotroph except without the deletion. Also provided is a corresponding non-naturally occurring methylotroph from whose genome (a) native rpe, (b) native rpe and native edd, (c) native rpe and native eda, or (d) native rpe, native edd and eda are deleted. This non-naturally occurring methylotroph may be prepared by deleting (a) native rpe, (b) native rpe and native edd, (c) native rpe and native eda, or (d) native rpe, native edd and eda from the genome of a non-naturally occurring methylotroph.

In yet another embodiment, the method comprises growing a non-naturally occurring methylotroph in a medium comprising methanol along with ribose or gluconate, where native ribulose-phosphate 3-epimerase gene (rpe), native phosphogluconate dehydratase gene (edd) and native 2-dehydro-3-deoxy-D-gluconate 6-phosphate (KDPG) aldolase gene (eda) are deleted from the genome of the non-naturally occurring methylotroph. The method may further comprise increasing production of a metabolite by the non-naturally occurring methylotroph by at least about 5%, 10%, 15%, 20%, 30%, 40%, 50%, 60%, 70%, 80%, 90%, 95%, 99%, 100% or 200% as compared with that by a control microbe, which is identical to the non-naturally occurring methylotroph except without the deletion. Also provided is a corresponding non-naturally occurring methylotroph from whose genome native rpe, native edd and eda are deleted. This non-naturally occurring methylotroph may be prepared by deleting native rpe, native edd and eda from the genome of a non-naturally occurring methylotroph.

In yet another embodiment, the method comprises growing a non-naturally occurring methylotroph in a medium comprising methanol, where native fructose-1,6-bisphosphatase class 1 gene (fbp), native fructose-1,6-bisphosphatase 1 class 2 gene (glpX), and one or more genes selected from the group consisting of (a) native 2,3-bisphosphoglycerate-dependent phosphoglycerate mutase gene (gpmA) and 2,3-bisphosphoglycerate-independent phosphoglycerate mutase gene (gpmM), (b) native glyceraldehyde-3-phosphate dehydrogenase gene (gapA) and glyceraldehyde-3-phosphate dehydrogenase gene (gapC), (c) native phosphoglycerate kinase gene (pgk) and (d) native enolase gene (eno) are deleted from the genome of the non-naturally occurring methylotroph. The method may further comprise increasing production of a metabolite by the non-naturally occurring methylotroph by at least about 5%, 10%, 15%, 20%, 30%, 40%, 50%, 60%, 70%, 80%, 90%, 95%, 99%, 100% or 200% as compared with that by a control microbe, which is identical to the non-naturally occurring methylotroph except without the deletion. Optionally, native methylglyoxal synthase gene (mgsA), native 6-phosphogluconate dehydrogenase gene (gnd) or native HTH-type transcriptional regulator GntR gene (gntR) may be deleted from the genome of the non-naturally occurring methylotroph.

Also provided is a corresponding non-naturally occurring methylotroph from whose genome (a) native fbp, native glpX, native gpmA and native gpmM, (b) native fbp, native glpX, native gapA and native gapC, (c) native fbp, native glpX and native pgk, or (d) native fbp, native glpX and native eno are deleted. Native mgsA, native gnd or native gntR may be optionally deleted from the genome of the non-naturally occurring methylotroph. The non-naturally occurring methylotroph may be prepared by deleting (a) native fbp, native glpX, native gpmA and native gpmM, (b) native fbp, native glpX, native gapA and native gapC, (c) native fbp, native glpX and native pgk, or (d) native fbp, native glpX and native eno, optionally along with native mgsA, native gnd or native gntR, from the genome of a non-naturally occurring methylotroph.

In yet another embodiment, the method comprises growing a non-naturally occurring methylotroph in a medium comprising methanol, glucose and serine, where (a) native aminomethyltransferase gene (gcvT), (b) native glycine cleavage system H protein gene (gcvH), (c) native glycine decarboxylase gene (gcvP), and (d) native phosphoglycerate dehydrogenase gene (serA) or serine hydroxymethyltransferase gene (glyA) are deleted from the genome of the non-naturally occurring methylotroph. The method may further comprise increasing production of a metabolite by the non-naturally occurring methylotroph by at least about 5%, 10%, 15%, 20%, 30%, 40%, 50%, 60%, 70%, 80%, 90%, 95%, 99%, 100% or 200% as compared with that by a control microbe, which is identical to the non-naturally occurring methylotroph except without the deletion. Also provided is a corresponding non-naturally occurring methylotroph from whose genome (a) native gcvT, native gcvH, native gcvP and native serA or (b) native gcvT, native gcvH, native gcvP and native glyA are deleted. The non-naturally occurring methylotroph may be prepared by deleting (a) native gcvT, native gcvH, native gcvP and native serA or (b) native gcvT, native gcvH, native gcvP and native glyA from the genome of a non-naturally occurring methylotroph.

In yet another embodiment, the method comprises growing a non-naturally occurring methylotroph in a medium comprising methanol along with glucose and glycine, where native aminomethyltransferase gene (gcvT), native glycine cleavage system H protein gene (gcvH), native glycine decarboxylase gene (gcvP) and native serine hydroxymethyltransferase gene (glyA) are deleted from the genome of the non-naturally occurring methylotroph while the non-naturally occurring methylotroph expresses heterologous formate-tetrahydrofolate ligase (FTL), heterologous methanol dehydrogenase (MDH), heterologous 3-hexulose-6-phosphate synthase (HPS), and heterologous 3-hexulose-6-phosphate isomerase (PHI). The method may further comprise increasing production of a metabolite by the non-naturally occurring methylotroph by at least about 5%, 10%, 15%, 20%, 30%, 40%, 50%, 60%, 70%, 80%, 90%, 95%, 99%, 100% or 200% as compared with that by a control microbe, which is identical to the non-naturally occurring methylotroph except without the deletion. Also provided is a corresponding non-naturally occurring methylotroph from whose genome native gcvT, native gcvH, native gcvP and native glyA are deleted and which expresses heterologous FTL, heterologous MDH, heterologous HPS, and heterologous PHI. The non-naturally occurring methylotroph may be prepared by deleting native gcvT, native gcvH, native gcvP and native glyA from the genome of a non-naturally occurring methylotroph and expressing heterologous FTL, heterologous MDH, heterologous HPS, and heterologous PHI.

In yet another embodiment, the method comprises growing a non-naturally occurring methylotroph in a medium comprising methanol along with glycerol or fructose, where native transaldolase A gene (talA), native transaldolase B gene (talB), native fructose-1,6-bisphosphatase class 1 gene (fbp) and native fructose-1,6-bisphosphatase 1 class 2 gene (glpX) are deleted. The method may further comprise increasing production of a metabolite by the non-naturally occurring methylotroph by at least about 5%, 10%, 15%, 20%, 30%, 40%, 50%, 60%, 70%, 80%, 90%, 95%, 99%, 100% or 200% as compared with that by a control microbe, which is identical to the non-naturally occurring methylotroph except without the deletion. Also provided is a corresponding non-naturally occurring methylotroph from whose genome native to/A, native talB, native fbp and native glpX are deleted. The non-naturally occurring methylotroph may be prepared by deleting native to/A, native talB, native fbp and native glpX from the genome of a non-naturally occurring methylotroph.

According to the method of the present invention, the Embden-Meyerhof-Parnas (EMP) pathway may be disrupted in the non-naturally occurring methylotroph. Disruption of the EMP pathway may be evidenced by methanol-dependent growth of the non-naturally occurring methylotroph, i.e., limited or negligible cell growth or co-substrate consumption in the absence of methanol and restored cell growth or co-substrate consumption in the presence of methanol. The advantages of disrupting the EMP pathway involve improving methanol consumption and the conversion of methanol-derived carbon into cell mass and metabolites over that of a cell not containing a disruption of the EMP pathway.

According to the method of the present invention, the Entner-Doudoroff (ED) pathway may be disrupted in the non-naturally occurring methylotroph. Disruption of the ED pathway may be evidenced by methanol-dependent growth of the non-naturally occurring methylotroph, i.e., limited or negligible cell growth or co-substrate consumption in the absence of methanol and restored cell growth or co-substrate consumption in the presence of methanol. The advantages of disrupting the ED pathway involve improving methanol consumption and the conversion of methanol-derived carbon into cell mass and metabolites over that of a cell not containing a disruption of the ED pathway.

Recombinant strains exhibiting methanol-dependent growth (i.e., non-naturally occurring methylotrophs) may be used as a tool for growth-based screening and selection of improved methylotrophic phenotypes. For example, homologs and/or libraries of essential methylotrophic genes (mdh, hps, phi), genomic gene deletion and/or knockdown libraries, and/or transposon and/or metagenomics libraries may be screened under methanol-dependent growth conditions to identify those library members exhibiting an improved methanol-dependent growth phenotype. More specifically, an improved MDH mutant methylotroph exhibiting a higher rate and/or specificity of methanol oxidation over that of its parent (i.e., non-mutant) MDH methylotroph may be identified based on an improved methanol-dependent growth rate due to, for example, an improved rate and/or specificity of methanol oxidation. Additionally, recombinant strains exhibiting methanol-dependent growth may be adaptively evolved for growth on methanol as the sole carbon source.

The method of the present invention may further comprise oxidizing the methanol. At least about 5%, 10%, 15%, 20%, 30%, 40%, 50%, 60%, 70%, 80%, 90%, 95%, 99% or 100% of the methanol in the medium may be oxidized.

The metabolites may be selected from the group consisting of 4-carbon chemicals, diacids, 3-carbon chemicals, higher carboxylic acids, alcohols of higher carboxylic acids, polyhydroxyalkanoates, and specialty chemicals. The 4-carbon chemicals may be selected from the group consisting of butyrate, n-butanol, i-butanol, 2-butanol, 1,3-butanediol, 2,3-butanediol, and 1,4-butanediol. The diacids may be selected from the group consisting of oxalic, malonic, succinic, glutaric, adipic, pimelic, pthalic, isopthalic, and terephtlalic. The 3-carbon chemicals may be selected from the group consisting of acetone, isopropanol, propanol, propanediol, lactate, 3-hydroxypropionate, and acrylate (acrylic acid). The higher carboxylic acids may be selected from the group consisting of pentanoic acids and hexanoic acids. Preferably, the metabolite is n-butanol. The specialty chemicals may include artemisinin, vanillin, anthocyanins and resveratrol.

The metabolite may be selected from the group consisting of acetone, isopropanol, 1,3-butanediol and n-butanol. Where the metabolite is acetone, the method of the present invention may further comprise expressing a heterologous enzyme having an acetyl-CoA C-acetyltransferase activity (e.g., thiolase (THL, EC 2.3.1.9)), a heterologous enzyme having a butyrate-acetoacetate CoA-transferase activity (e.g., acyl(acetate/butyrate)-acetoacetate coenzyme A transferase (CTFA/B, EC 2.8.3.9)) and a heterologous enzyme having acetoacetate decarboxylase activity (e.g., acetoacetate decarboxylase (ADC, EC 4.1.1.4)) in the non-naturally occurring methylotroph.

The method may further comprise incorporating a carbon atom from the methanol into one or more metabolites. At least about 30%, 40%, 50%, 60%, 70%, 80%, 90% or 100%, preferably at least about 80%, of the carbon in the metabolite is derived from the methanol. In some embodiments, the metabolite is an amino acid or tricarboxylic acid (TCA) intermediate having at one or multiple carbon positions of the chemical up to the fourth position derived from the methanol. The present method may produce a desirable metabolite at about 50-100 mg.

The methanol carbon may be incorporated throughout central metabolism and into biomass of the non-naturally occurring methylotroph. For example, the methanol carbon has been found in RNA molecules via, for example, the PPP pathway, glycogen and pyruvate via, for example, the EMP pathway, tricarboxylic acids (e.g., citrate) and amino acids via, for example, the TCA cycle. The methanol carbon has also been found in acetyl-CoA derived metabolites (e.g., acetone, 1-butanol). Since most metabolites are derived from pyruvate and/or acetyl-CoA, the majority of the metabolites (if not all) can be produced from methanol.

According to the method of the present invention, the non-naturally occurring methylotroph may be grown aerobically, microaerobically or anaerobically. Under the aerobic condition, the medium contains more than 10% of the oxygen dissolvable in water under the same conditions. Under the microaerobic condition, the medium contains less than 10% of the oxygen dissolvable in water under the same conditions.

According to the method of the present invention, the non-naturally occurring methylotroph may be grown at a temperature of at least about 30° C. or about 37° C., for example, at about 30° C., 37° C., 40° C., 45° C. or 50° C., or in a range from about 30° C. to about 37° C. In one embodiment, the non-naturally occurring methylotroph may be grown at a temperature in a range from about 30° C. to about 37° C. In another embodiment, the non-naturally occurring methylotroph may be grown at a temperature of about 30° C. In yet another embodiment, the non-naturally occurring microbe may be grown at a temperature of about 37° C.

A gene encoding a heterologous enzyme, for example, MDH, the RuMP pathway enzymes (e.g., HPS and PHI), the PPP pathway enzymes (e.g., PFK, FBA, TKT, TAL, GLPX, RPI, and RPE), the cyclic formaldehyde dissimilation enzymes (e.g., PGI, ZWF, PGL, and GND), the CO₂ fixation pathway enzymes (e.g., CA, FDH, FLD, reductive tricarboxylic acid cycle enzymes such as ACL, OGOR, ICDH, and FR, glycine cleavage system enzymes such as AMT, LPDH, GDH, non-oxidative glycolysis pathway enzymes such as fructose phosphoketolase, xylose phosphoketolase, transaldolase, transketolase, fructose 1,2-bisphosphate aldolase, fructose 1,6-bisphosphatase, ribulose-5-phosphate epimerase, ribose-5-phosphate isomerase, and triose phosphate isomerase, DHAS, and DAK, may be modified to improve metabolite production, methanol oxidization or methanol utilization. The gene may be engineered to be under control of an inducible promoter, for example, a formaldehyde or methanol responsive promoter, a lactose inducible promoter, or a temperature or pH responsive promoter. These promoters may be derived from a host cell (native) or exogenously, for example, the T7 phage promoter. These genes may also be under the control of non-DNA regulatory elements such as small RNA, antisense RNA, sensing RNA, temperature sensitive RNA or any combination thereof. The translation of these genes may be initiated with a range of ribosomal binding sites of varying strength. These genes may be borne on plasmids, fosmids, bacterial artificial chromosomes or be integrated into the host chromosome. These genes may be configured monocistronically or polycistronically. The gene may also be engineered to modify the corresponding enzyme (e.g., MDH) to improve the enzyme's substrate specificity and optimal temperature in the non-naturally occurring microbe.

The method may further comprise fixing CO₂. The medium may be modified by containing higher levels of methanol, which is more reduced than a sugar (e.g., glucose), such that more electrons may be generated under the conditions the non-naturally occurring microbe is grown. Other medium modifications may also enable an enhanced availability of electrons in the cells. Such additives would be reducing agents or dyes (such as Methyl Viologen (MV) and other viologens). Such electrons may enable the non-naturally occurring methylotroph to grow on the medium while fixing CO₂. According to this method, CO₂ release may be reduced by at least about 20%, preferably by at least about 30-50%, more preferably up to about 75%.

According to the method of the present invention, the methanol may contribute to a significant percentage of the carbon source in the medium for the non-naturally occurring methylotroph. The methanol may contribute to at least about 40%, 48%, 50%, 60%, 66%, 70%, 80%, 90%, 95%, 99%, or 100% of the carbon source, based on weight, for the non-naturally occurring methylotroph. Preferably, the methanol may contribute to at least about 40% of the carbon source. More preferably, the methanol is the sole carbon source, i.e., contributing 100% of the carbon source, for non-naturally occurring methylotroph.

According to the method of the present invention, the medium may further comprise one or more other carbon sources, for example, fermentable mono-, di-, oligo- or polysaccharides. Exemplary fermentable monosaccharides include glucose, xylose, mannose, arabinose, rhamnose, and ribose. Fermentable di- or oligosaccharides may be sucrose, lactose, maltose, cellobiose, short polymers of these mono- or di-saccharides, or long polymers of saccharides, for example, cellulose and xylan. The medium may further comprise other carbon source, for example, amino acids. Exemplary amino acids include alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine. The other carbon source may contribute to no more than about 40%, preferably no more than about 30%, more preferably no more than about 20%, most preferably no more than about 10% of the carbon source for the non-naturally occurring methylotroph.

The term “about” as used herein when referring to a measurable value such as an amount, a percentage, and the like, is meant to encompass variations of ±20% or ±10%, more preferably ±5%, even more preferably ±1%, and still more preferably ±0.1% from the specified value, as such variations are appropriate.

### EXAMPLES

### Example 1

Genetic modifications, such as gene deletions and/or overexpression of native and/or heterologous genes, enhance methanol assimilation and methanol and/or formaldehyde carbon flux in cells when exposed to methanol and/or formaldehyde as substrates.

Genetic modifications may comprise gene deletions, gene knockdowns, and/or gene overexpressions, and/or a combination thereof. Gene deletions may be performed following standard homologous recombination protocols that rely on native and/or phage-derived recombination machinery. Gene knockdowns may be performed following standard transcription and/or translation interference. CRISPRi (CRISPR interference) involves interference of transcription via the action of dCas9 (dead Cas9) binding specifically to a gene locus via a gRNA (guide RNA), which limits RNA polymerase binding that results in reduced gene expression. Translational interference may be performed via siRNA (small interfering RNA) that bind specifically to an mRNA (messenger RNA) transcript, which limits ribosome binding that results in reduced gene expression. Gene overexpression may be performed following standard protocols that include gene knockins via homologous recombination (similar to gene deletions above), increased copy number of the gene that is to be overexpressed, increased promoter and/or RBS strengths, and/or CRISPRa (CRISPR activation). Analytical methods to assess methylotrophic phenotypes may include, but are not limited to, spectrophotometry (to determine biomass concentrations and ¹³C metabolite labeling) and chromatography (e.g. liquid or gas to determine metabolite concentrations).

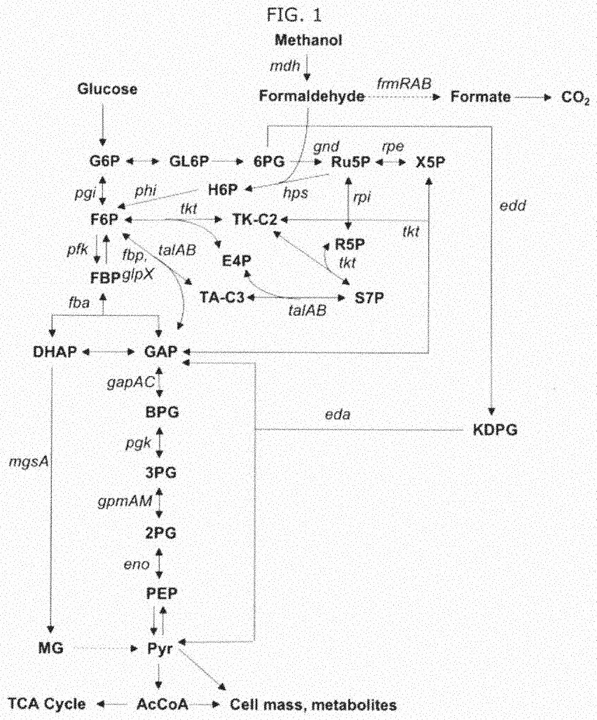

Deletion of native genes, such as glucose-6-phosphate isomerase (pgi), phosphogluconate dehydratase (edd), 2-dehydro-3-deoxy-D-gluconate 6-phosphate (KDPG) aldolase (eda), leucine-responsive regulatory protein (lrp), GDP pyrophosphokinase/GTP pyrophosphokinase (relA), bifunctional (p)ppGpp synthetase II/guanosine-3′,5′-bis pyrophosphate 3′-pyrophosphohydrolase (spoT) or RNA polymerase-binding transcription factor (dksA), enhance methanol assimilation and the methanol and/or formaldehyde carbon flux in cells when exposed to methanol and/or formaldehyde as substrates. Deletion of the glucose-6-phosphate isomerase (pgi) gene conserves methanol carbon by forcing methanol flux through glycolysis and away from the oxidative pentose phosphate pathway, which results in carbon loss to CO₂ (FIG. 1). Deletion of the glucose-6-phosphate isomerase (pgi) and phosphogluconate dehydratase (edd) genes provides sustained regeneration of ribulose-5-phosphate (Ru5P) from glucose, which is forced through the oxidative pentose phosphate pathway, to sustain methanol assimilation (FIG. 1).

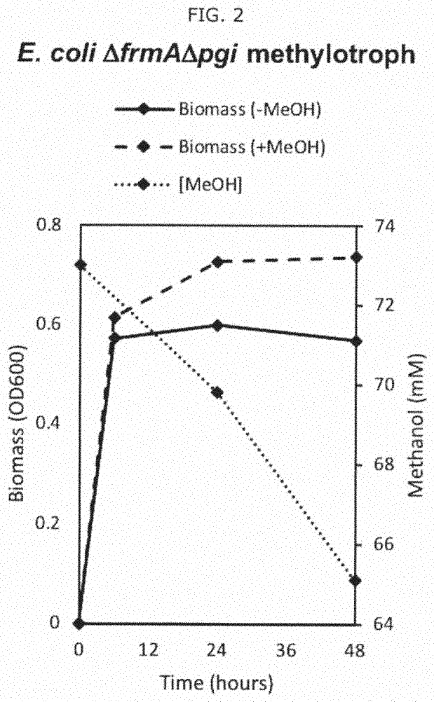

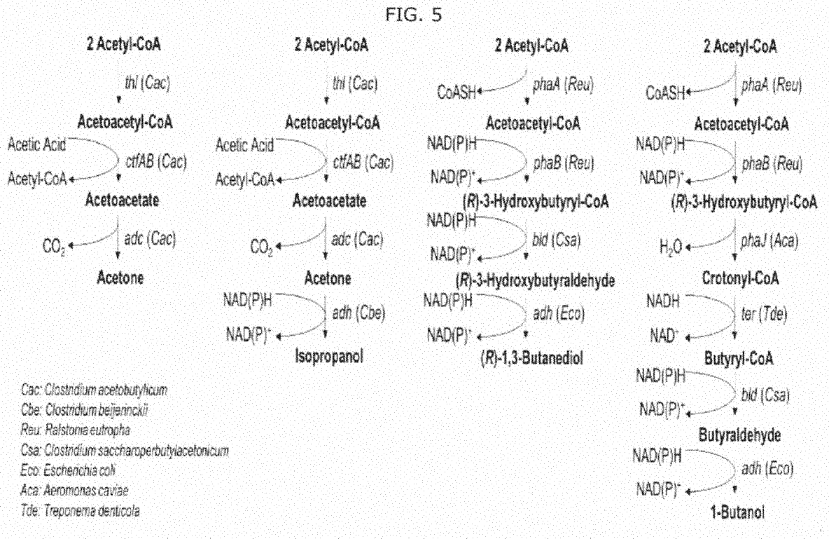

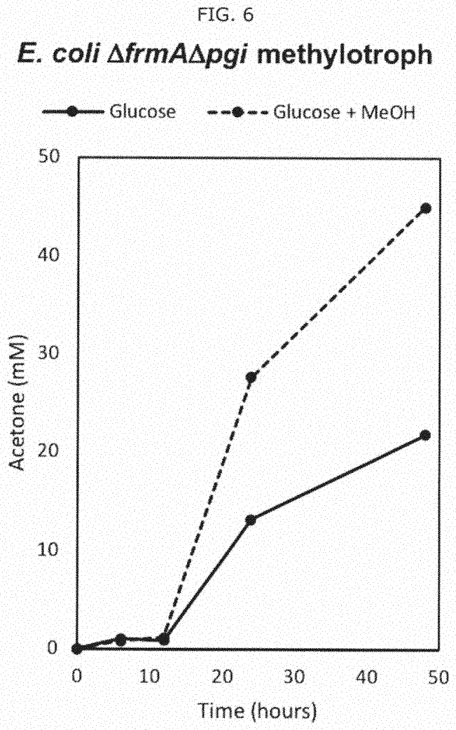

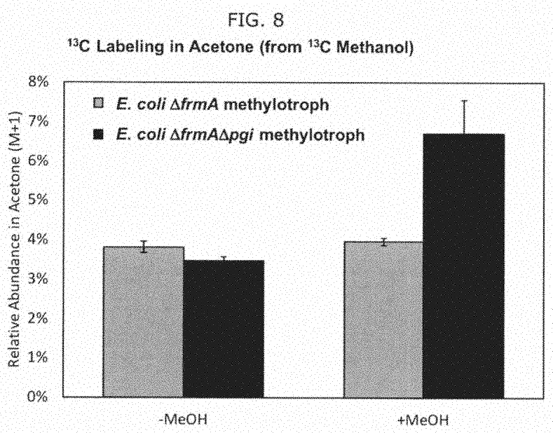

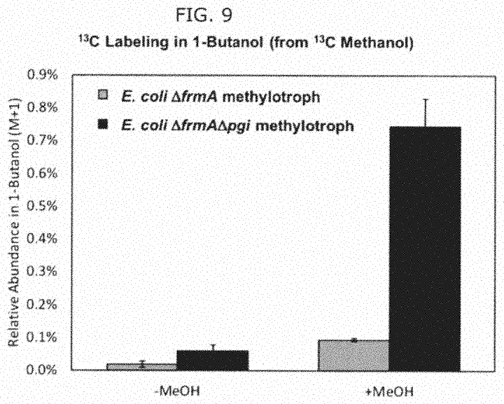

As described, deletion of the glucose-6-phosphate isomerase (pgi) gene forces glucose carbon flux through the oxidative pentose phosphate pathway to provide sustained regeneration of ribulose-5-phosphate (Ru5P), which is required for sustaining methanol assimilation (FIG. 2). During acetone (or n-butanol) fermentation, this co-utilization of glucose and methanol enhances acetone (or n-butanol) titer when compared to the control culture absent of methanol supplementation (FIGS. 6, 8, 9). Expression of heterologous genes allow E. coli to produce acetone (or n-butanol) from acetyl-CoA (FIG. 5). Acetoacetate decarboxylase is encoded by adc, acetoacetate CoA-transferases are encoded by ctfA and ctfB, and thiolase is encoded by thl.

E. coli primarily uses the Embden-Meyerhof-Parnas (EMP) pathway (i.e. glycolysis) for glucose catabolism. Pgi (glucose-6-phosphate isomerase, encoded by pgi) is an enzyme that catalyzes an initial step in the EMP pathway, that of the following (Eq. 3):

D-glucopyranose 6-phosphate (G6P)→β-D-fructofuranose 6-phosphate (F6P) (3)

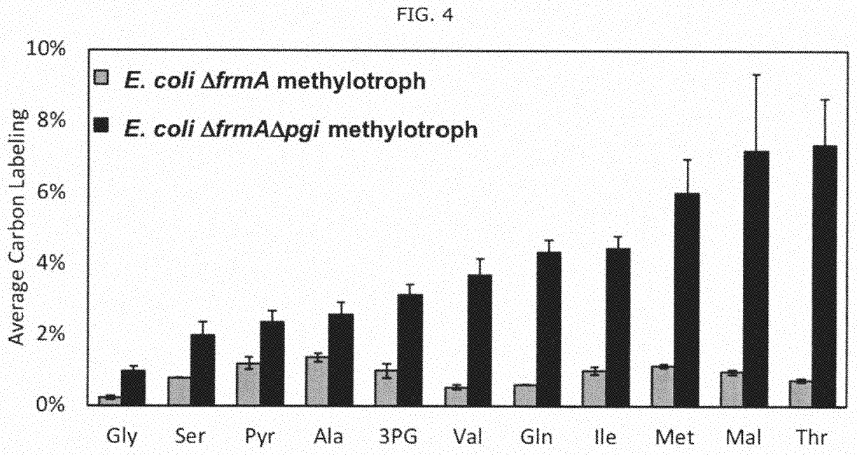

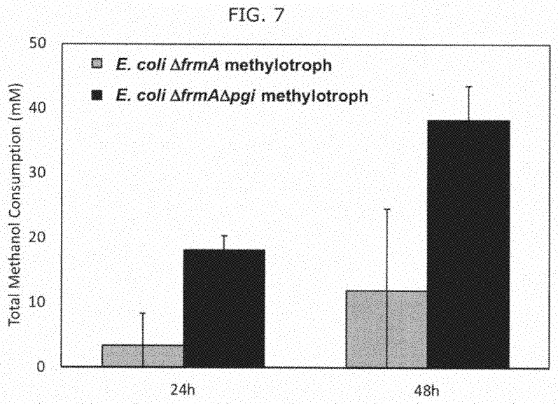

As the primary pathway for glucose catabolism, the EMP pathway carries the majority of glucose carbon flux, which limits the amount of carbon flux through the pentose phosphate pathway (PPP) for ribulose 5-phosphate (Ru5P) production. Since Ru5P is an essential substrate of hps for formaldehyde fixation (i.e. hps catalyzes the conversion of: formaldehyde+Ru5P→hexulose 6-phosphate (H6P)), its production is important for formaldehyde assimilation into central carbon metabolism. To improve methanol consumption in the presence of glucose (i.e. the co-utilization of methanol and glucose), deletion of pgi may be performed. Deletion of pgi will re-route glucose carbon flux away from the EMP pathway and through the oxidative PPP toward Ru5P. In this way, Ru5P production is improved in E. coli Δpgi compared to wild-type E. coli. Since Ru5P production is improved in E. coli Δpgi, methanol consumption is also improved in E. coli Δpgi compared to wild-type E. coli (FIGS. 4, 7, 8, 9). In addition to increasing the oxidative PPP carbon flux in E. coli Δpgi, the carbon flux through the Entner-Doudoroff (ED) pathway also increases. The ED pathway, consisting of the genes edd (phosphogluconate dehydratase) and eda (2-dehydro-3-deoxy-D-gluconate 6-phosphate (KDPG) aldolase), catalyze the following reactions (Eqs. 4-5):

D-gluconate 6-phosphate (6PG)→KDPG+H₂O (4)

KDPG D-glyceraldehyde 3-phosphate (GAP)+pyruvate (5)

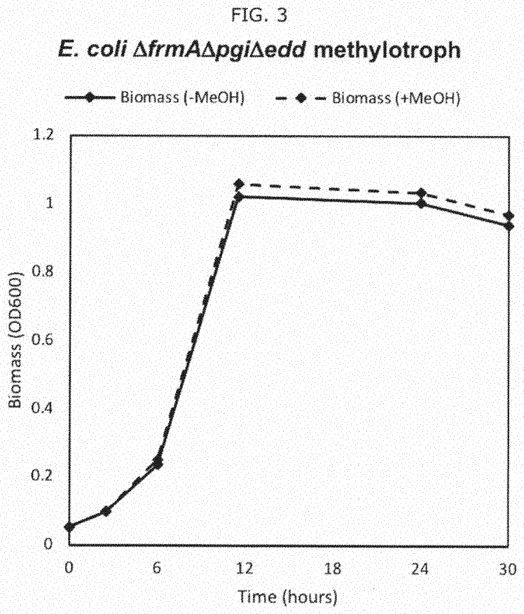

Since the ED pathway acts on the metabolite 6PG, which is one step prior to Ru5P production via gnd (6-phosphogluconate dehydrogenase), consumption of 6PG via the ED pathway reduces the amount of carbon flux toward Ru5P production. Therefore, deletion of either edd or eda, alone or in combination, may improve the carbon efficiency of carbon flux toward Ru5P production, thus increasing Ru5P production and ultimately methanol consumption (FIG. 3). This strategy improves the co-utilization of methanol and glucose in a synthetic methylotroph. Furthermore, while these gene deletions have been performed and characterized in native E. coli, it is a new strategy of deleting pgi, edd, or eda, alone or in combination, to improve the co-utilization of methanol and glucose in the context of synthetic methylotroph.

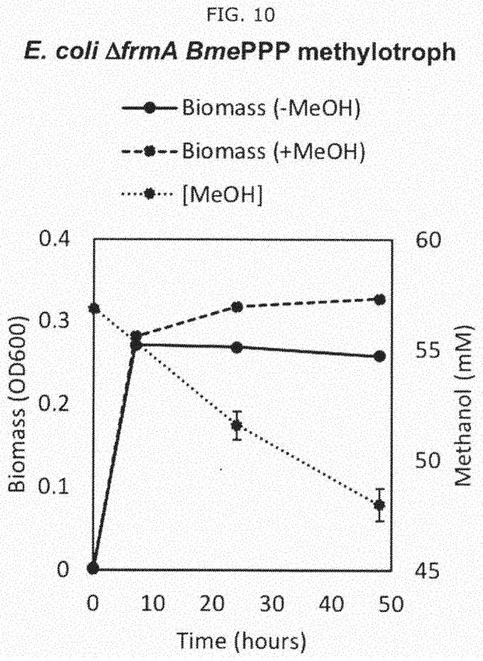

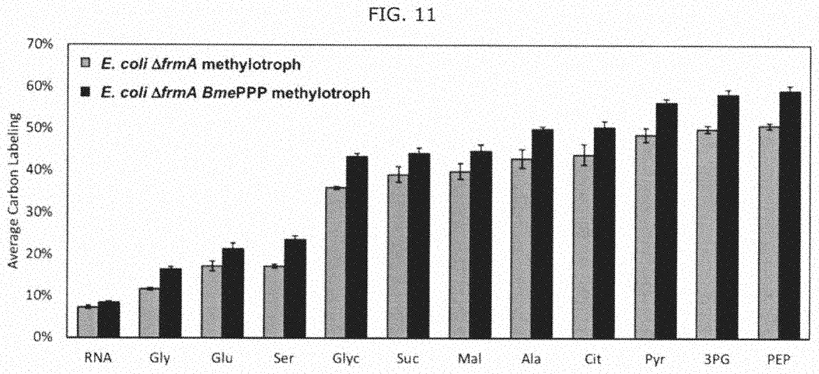

Expressing one or more heterologous pentose-phosphate pathway (PPP) enzymes in the non-methylotrophic microbe improves PPP activity and ribulose 5-phosphate (Ru5P) production. The one or more heterologous PPP enzymes may comprise heterologous phosphofructokinase (PFK), heterologous fructose bisphosphate aldolase (FBA), heterologous transketolase (TKT), transaldolase (TAL), heterologous fructose/sedoheptulose bisphosphatase (GLPX), heterologous ribose-5-phosphate isomerase (RPI), and heterologous ribulose phosphate epimerase (RPE). As described, expression of these heterologous PPP genes improve PPP activity in E. coli to produce more Ru5P for improved formaldehyde assimilation (FIGS. 10, 11).

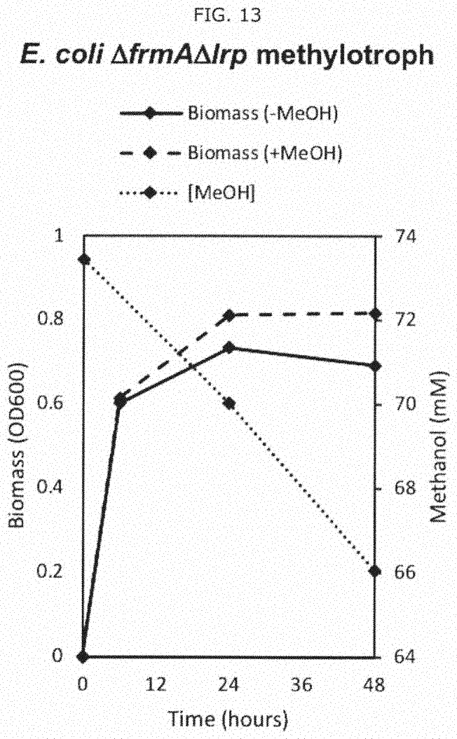

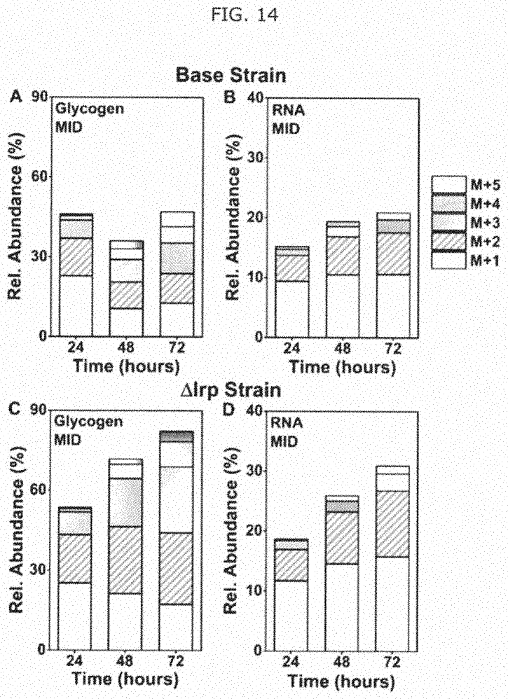

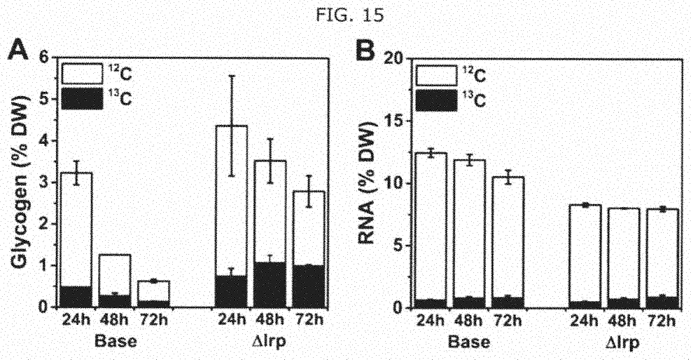

Deletion of the gene (lrp) coding for the leucine-responsive regulatory protein (LRP) affects the expression of many genes (FIG. 12). Deletion of the leucine-responsive regulatory protein (lrp) gene enhances biomass yield and ¹³C labeling in glycogen and RNA (derived from ¹³C-methanol) in a nutritionally limited media by combatting the native starvation stress response of Escherichia coli (FIGS. 13, 14, 15). LRP levels in Escherichia coli are directly correlated with guanosine pentaphosphate (ppGpp) levels. Therefore, the deletion of GDP pyrophosphokinase/GTP pyrophosphokinase (relA), bifunctional (p)ppGpp synthetase II/guanosine-3′,5′-bis pyrophosphate 3′-pyrophosphohydrolase (spoT) and/or RNA polymerase-binding transcription factor (dksA), which are all involved in the biosynthesis of ppGpp, is thought to also enhance methanol assimilation in a synthetic methylotrophic organism.

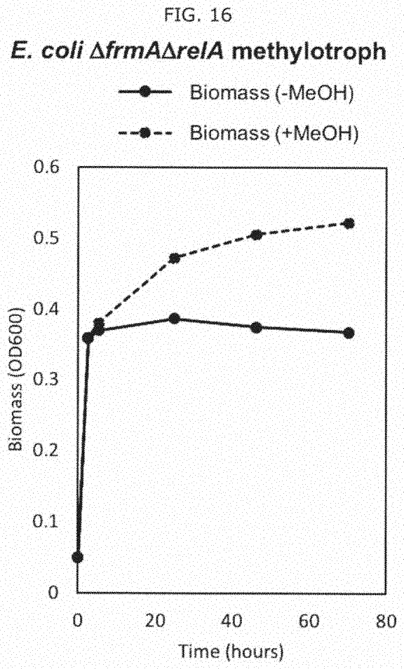

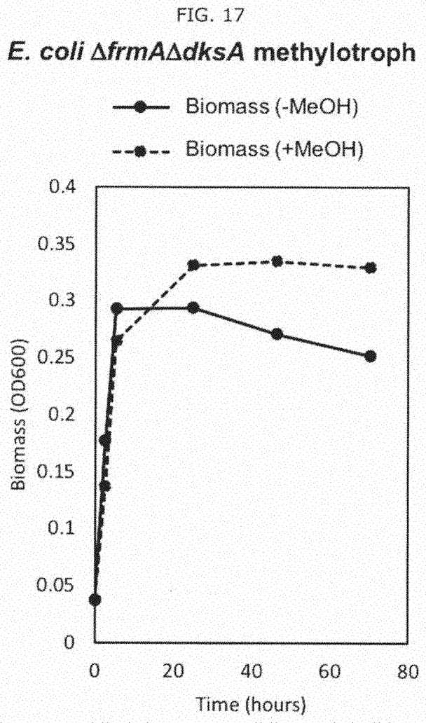

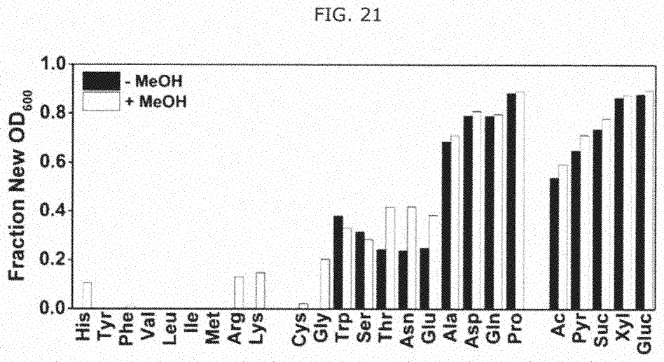

Yeast extract stimulates methanol consumption and growth on methanol in a synthetic methylotroph. Yeast extract is primarily composed of amino acids in the form of peptides (i.e., a chain having 2-10 amino acids) and polypeptides (i.e., a chain having more than 10 amino acids). To elucidate the importance of each amino acid in the context of synthetic methylotrophy, methanol consumption and growth on methanol of a synthetic E. coli methylotroph were analyzed in minimal medium containing ¹³C-methanol and one of the 20 standard amino acids (FIG. 21). For example, one experiment contained ¹³C-methanol and alanine, while a separate experiment contained ¹³C-methanol and threonine, etc. In comparison to growth in minimal medium containing ¹³C-methanol and glucose (which resulted in low methanol consumption), growth in minimal medium containing ¹³C-methanol and threonine resulted in high methanol consumption. In E. coli threonine is catabolized in the direction of threonine→glycine→serine (FIG. 12). The opposite direction occurs during growth on glucose (i.e. serine→glycine→threonine) to produce threonine from glucose. Therefore, the flux from threonine→glycine→serine during threonine catabolism has an impact on methanol consumption since high methanol consumption is observed in the presence of threonine but not glucose. Furthermore, threonine catabolism is regulated by lrp (encoding the leucine-responsive regulatory protein). Natively, Lrp acts to down-regulate the desired flux from threonine→glycine→serine. To alleviate this undesired down-regulation, lrp may be deleted from the genome of E. coli. Deletion of lrp resulted in improved methanol consumption compared to lrp-intact E. coli. Additionally, the levels of lrp are directly correlated with guanosine pentaphosphate (ppGpp) levels. ppGpp, an alarmone, is involved in the bacterial stringent response, resulting in RNA synthesis inhibition when amino acid levels are low. Therefore, deletion of GDP pyrophosphokinase/GTP pyrophosphokinase (relA), bifunctional (p)ppGpp synthetase II/guanosine-3′,5′-bis pyrophosphate 3′-pyrophosphohydrolase (spoT) and/or RNA polymerase-binding transcription factor (dksA), which are all involved in the biosynthesis of ppGpp, may also enhance methanol consumption in a synthetic methylotroph (FIGS. 16, 17). Deletion of relA, spoT, and/or dksA are expected to mimic the effect that the deletion of lrp has on methanol consumption since low ppGpp levels will result in low lrp levels. This strategy improves methanol consumption in a synthetic methylotroph. Furthermore, while these gene deletions have been performed and characterized in native E. coli, it is a new strategy of deleting lrp, relA, spoT, or dksA, alone or in combination, to improve methanol consumption in the context of synthetic methylotrophy.

Deletion of additional native genes, such as ribulose-phosphate 3-epimerase (rpe), transaldolase A (talA), transaldolase B (talB), phosphoglycerate dehydrogenase (serA), serine hydroxymethyltransferase (glyA), aminomethyltransferase (gcvT), glycine cleavage system H protein (gcvH), or glycine decarboxylase (gcvP), may further enhance methanol assimilation and the methanol and/or formaldehyde carbon flux in cells when exposed to methanol and/or formaldehyde as substrates. Deletion of one or more of these native genes may allow enhanced methanol and/or formaldehyde assimilation in the absence and/or presence of additional growth substrates, including, but not limited to, monosaccharides and/or amino acids.

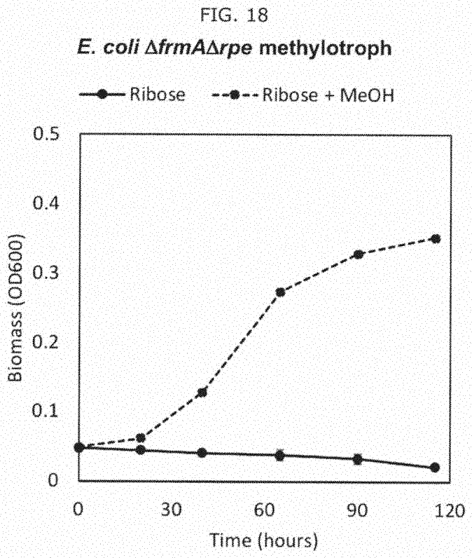

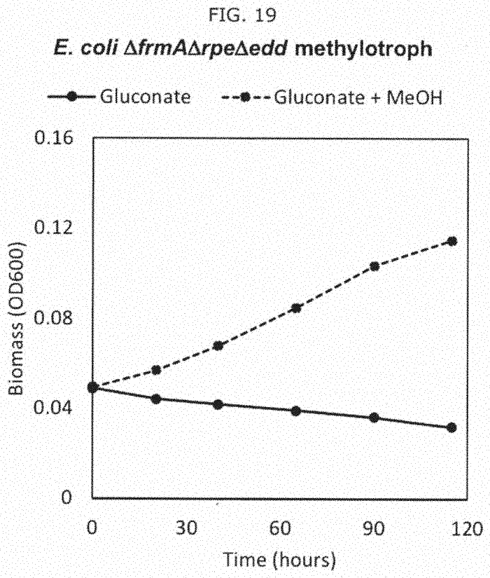

Gene deletions that allow methanol-dependent growth on native co-substrates were identified. For example, deletion of rpe (ribulose-phosphate 3-epimerase) renders wild-type E. coli unable to metabolize and thus grow on ribose minimal medium. Rpe catalyzes the interconversion of Ru5P and xylulose 5-phosphate (X5P). During ribose catabolism, ribose is initially phosphorylated to ribose 5-phosphate (R5P) and subsequently converted to Ru5P via rpiAB (R5P isomerase). In E. coli Δrpe, this is where ribose catabolism ends since Ru5P cannot be converted to X5P due to deletion of rpe, thus growth on ribose does not occur. To rescue this non-growth phenotype, the synthetic methanol consumption pathway converts Ru5P (with formaldehyde) to F6P, which is then able to supply all required metabolites for growth (FIG. 18). As a result, this is termed methanol-dependent growth, in this case on ribose. Similarly, deletion of edd or eda, alone or in combination, in addition to the rpe deletion renders wild-type E. coli unable to metabolize and thus grow on gluconate minimal medium. Gluconate catabolism begins with phosphorylation to 6PG, which can subsequently be consumed by gnd in the PPP or the ED pathway. To eliminate consumption via the ED pathway, deletion of edd or eda, alone or in combination, may be performed. Deletion of deletion of edd or eda, alone or in combination, in addition to the rpe deletion allows gluconate to be metabolized only to the point of R5P and thus unable to grow. As above, to rescue this non-growth phenotype, the synthetic methanol consumption pathway converts Ru5P (with formaldehyde) to F6P, which is then able to supply all required metabolites for growth (FIG. 19). As a result, this is also termed methanol-dependent growth, in this case on gluconate. This strategy not only improves methanol consumption in a synthetic methylotroph but it allows growth in the presence of methanol whereas no growth occurs in the absence of methanol. Furthermore, while these gene deletions have been performed and characterized in native E. coli, it is a new strategy of deleting rpe, edd, or eda, alone or in combination, to improve methanol consumption and allow methanol-dependent growth in the context of synthetic methylotrophy.

Deletion of additional native genes, such as 2,3-bisphosphoglycerate-dependent phosphoglycerate mutase (gpmA), 2,3-bisphosphoglycerate-independent phosphoglycerate mutase (gpmM), glyceraldehyde-3-phosphate dehydrogenase (gapA), glyceraldehyde-3-phosphate dehydrogenase (gapC), phosphoglycerate kinase (pgk), enolase (eno), fructose-1,6-bisphosphatase class 1 (fbp), fructose-1,6-bisphosphatase 1 class 2 (glpX), methylglyoxal synthase (mgsA), 6-phosphogluconate dehydrogenase (gnd), HTH-type transcriptional regulator GntR (gntR) or S-(hydroxymethyl)glutathione dehydrogenase (frmA), may further enhance methanol assimilation and the methanol and/or formaldehyde carbon flux in cells when exposed to methanol and/or formaldehyde as substrates. Deletion of one or more of these native genes may allow enhanced methanol and/or formaldehyde assimilation in the absence and/or presence of additional growth substrates, including, but not limited to, monosaccharides and/or amino acids.

The combinations of the following gene deletions are expected to yield another methanol-dependent growth phenotype: fructose-1,6-bisphosphatase class 1 (fbp), fructose-1,6-bisphosphatase 1 class 2 (glpX), methylglyoxal synthase (mgsA), and either 2,3-bisphosphoglycerate-dependent phosphoglycerate mutase (gpmA) and 2,3-bisphosphoglycerate-independent phosphoglycerate mutase (gpmM), or glyceraldehyde-3-phosphate dehydrogenase (gapA) and glyceraldehyde-3-phosphate dehydrogenase (gapC), phosphoglycerate kinase (pgk), or enolase (eno). Deletion of either gpmA and gpmM, gapA and gapC, pgk, or eno acts to completely disrupt the EMP pathway. Deletion of fbp and glpX act to disrupt the gluconeogenic reaction of fructose 1,6-bisphosphate (FBP) to F6P. Deletion of mgsA further disrupts an EMP pathway bypass that converts dihydroxyacetone phosphate (DHAP) to pyruvate. In this way, catabolism of methanol must occur via the PPP and ED pathway in a synthetic methylotroph to supply all required metabolites for growth (e.g. TCA cycle intermediates). Additionally, deletions of 6-phosphogluconate dehydrogenase (gnd) and HTH-type transcriptional regulator GntR (gntR) may be performed to improve carbon flux through the ED pathway since gnd competes with the ED pathway for 6PG and gntR down-regulates the ED pathway. This strategy allows growth in the presence of methanol of a synthetic methylotroph whereas no growth occurs in the absence of methanol. Furthermore, while these gene deletions have been performed and characterized in native E. coli, it is a new strategy of deleting gpmA, gpmM, fbp, glpX, gnd, gntR, mgsA, gapA, gapC, pgk, eno, in distinct combinations, to allow methanol-dependent growth in the context of synthetic methylotrophy.

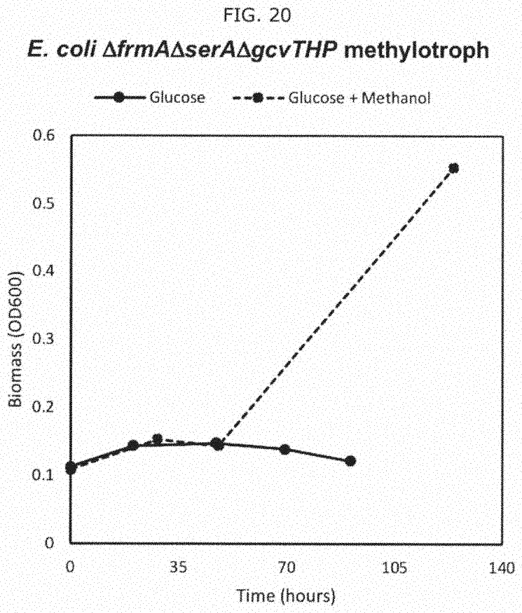

The combinations of the following gene deletions are expected to yield another methanol-dependent growth phenotype: aminomethyltransferase (gcvT), glycine cleavage system H protein (gcvH), glycine decarboxylase (gcvP), and either phosphoglycerate dehydrogenase (serA) or serine hydroxymethyltransferase (glyA). One strain contains deletions of serA, gcvT, gcvH, and gcvP, which requires serine supplementation for growth on glucose. However, after the overexpression of several additional genes, serine supplementation can be replaced by formate supplementation. We further manipulated this strain by introducing the synthetic methanol consumption pathway, which now allows methanol to substitute for formate, resulting in methanol-dependent growth on glucose minimal medium (FIG. 20). Separately, another strain contains deletions of glyA, gcvT, gcvH, and gcvP, which would also allow the same methanol-dependent growth phenotype. This strategy allows growth in the presence of methanol of a synthetic methylotroph whereas no growth occurs in the absence of methanol. Furthermore, while these gene deletions have been performed and characterized in native E. coli, it is a new strategy of deleting gpmA, gpmM, fbp, glpX, gnd, gntR, mgsA, gapA, gapC, pgk, eno, in distinct combinations, to allow methanol-dependent growth in the context of synthetic methylotrophy.

The combinations of the following gene deletions are expected to yield another methanol-dependent growth phenotype: transaldolase A (talA), transaldolase B (talB), fructose-1,6-bisphosphatase class 1 (fbp), and fructose-1,6-bisphosphatase 1 class 2 (glpX). These gene deletions, in combination, along with the synthetic methanol consumption pathway will allow methanol-dependent growth on glycerol or fructose. This strategy allows growth in the presence of methanol of a synthetic methylotroph whereas no growth occurs in the absence of methanol. Furthermore, while these gene deletions have been performed and characterized in native E. coli, it is a new strategy of deleting to/A, to/B, fbp, and glpX to allow methanol-dependent growth in the context of synthetic methylotrophy.

### Example 2. A Variety of Substrates, Including Individual Amino Acids, Enhance Growth and Methanol Assimilation in Methylotrophic Escherichia coli

As a co-substrate, methanol has been shown to enhance growth, biomass yield and metabolite labeling in methylotrophic Escherichia coli. Methanol has been used as a co-substrate with a variety of traditional growth substrates, including individual amino acids (FIG. 21). Among the amino acids, asparagine, glutamic acid and threonine exhibit the largest improvement in biomass yield with methanol supplementation compared to the control cultures absent of methanol. These data agree well with the finding that yeast extract, which is primarily composed of amino acids, stimulates methanol assimilation in Escherichia coli (FIGS. 2, 10).

All documents, books, manuals, papers, patents, published patent applications, guides, abstracts, and/or other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

1. A method for increasing production of a metabolite by a non-naturally occurring methylotroph, comprising growing the non-naturally occurring methylotroph in a medium comprising methanol, wherein expression of one or more native genes in the non-naturally occurring methylotroph is changed.

2. The method of claim 1, wherein the one or more native genes comprise 2,3-bisphosphoglycerate-dependent phosphoglycerate mutase gene (gpmA), 2,3-bisphosphoglycerate-independent phosphoglycerate mutase gene (gpmM), 2-dehydro-3-deoxy-D-gluconate 6-phosphate (KDPG) aldolase gene (eda), 6-phosphogluconate dehydrogenase gene (gnd), aminomethyltransferase gene (gcvT), bifunctional (p)ppGpp synthetase II/guanosine-3′,5′-bis pyrophosphate 3′-pyrophosphohydrolase gene (spoT), enolase gene (eno), fructose-1,6-bisphosphatase 1 class 2 gene (glpX), fructose-1,6-bisphosphatase class 1 gene (fbp), GDP pyrophosphokinase/GTP pyrophosphokinase gene (relA), glucose-6-phosphate isomerase gene (pgi), glyceraldehyde-3-phosphate dehydrogenase gene (gapA), glyceraldehyde-3-phosphate dehydrogenase gene (gapC), glycine cleavage system H protein gene (gcvH), glycine decarboxylase gene (gcvP), HTH-type transcriptional regulator GntR gene (gntR), leucine-responsive regulatory protein gene (lrp), methylglyoxal synthase gene (mgsA), phosphogluconate dehydratase gene (edd), phosphoglycerate dehydrogenase gene (serA), phosphoglycerate kinase gene (pgk), ribulose-phosphate 3-epimerase gene (rpe), RNA polymerase-binding transcription factor gene (dksA), serine hydroxymethyltransferase gene (glyA), transaldolase A gene (talA), transaldolase B gene (talB), or a combination thereof.

3. The method of claim 1, wherein the one or more native genes are deleted from the genome of the non-naturally occurring methylotroph, wherein the one or more native genes comprise leucine-responsive regulatory protein gene (lrp).

4. The method of claim 1, wherein the one or more native genes comprise one or more deletions.

5. The method of claim 1, wherein the medium further comprises a co-substrate.

6. The method of claim 5, wherein the co-substrate comprises one or more monosaccharides selected from the group consisting of glucose, xylose, mannose, arabinose, rhamnose, ribose and a combination thereof.

7. The method of claim 5, wherein the co-substrate comprises one or more amino acids selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine and a combination thereof.

8. The method of claim 1, wherein the non-naturally occurring methylotroph is a microbe derived from a microbe selected from the group consisting of Acetobacter, Acinetobacter, Bacillus, Chlorobi, Clostridium, Corynebacterium, Cyanobacteria, Deinococcus, Enterobacter, Enterobacteria, Escherichia, Geobacillus, Geobacter, Klebsiella, Lactobacillus, Lactococcus, Mannheimia, Propionibacterium, Pseudomonas, Ralstonia, Shewanella, Streptococcus, Streptomyces, Synechococcus, Synechocystis and Zymomonas.

9. The method of claim 1, wherein the non-naturally occurring methylotroph is Escherichia coli.

10. The method of claim 1, further comprising incorporating a carbon atom from the methanol into the metabolite.

11. The method of claim 1, wherein the metabolite is selected from the group consisting of 4-carbon chemicals, diacids, 3-carbon chemicals, higher carboxylic acids [which may include acetic acid, propionic acid, butyric acid, valeric (pentanoic) acid and caproic (hexanoic) acid], alcohols of higher carboxylic acids and polyhydroxyalkanoates.

12. The method of claim 1, wherein the metabolite is selected from the group consisting of acetone, isopropanol, 1,3-butanediol and n-butanol.

13. The method of claim 1, wherein the metabolite is acetone, further comprising expressing a heterologous enzyme having an acetyl-CoA C-acetyltransferase activity [e.g., thiolase (EC 2.3.1.9) (THL)], a heterologous enzyme having a butyrate-acetoacetate CoA-transferase activity [e.g., acyl(acetate/butyrate)-acetoacetate coenzyme A transferase (EC 2.8.3.9) (CTFA/B), and a heterologous enzyme having acetoacetate decarboxylase activity [e.g., acetoacetate decarboxylase (EC 4.1.1.4) (ADC) in the non-naturally occurring methylotroph.

14. The method of claim 1, further comprising increasing the production of the metabolite by the non-naturally occurring methylotroph by at least 5% as compared with that by a control microbe in which the expression of the one or more native genes is not changed.

15. A non-naturally occurring methylotroph, wherein expression of one or more native genes in the non-naturally occurring methylotroph is changed, wherein the one or more native genes comprise 2,3-bisphosphoglycerate-dependent phosphoglycerate mutase gene (gpmA), 2,3-bisphosphoglycerate-independent phosphoglycerate mutase gene (gpmM), 2-dehydro-3-deoxy-D-gluconate 6-phosphate (KDPG) aldolase gene (eda), 6-phosphogluconate dehydrogenase gene (gnd), aminomethyltransferase gene (gcvT), bifunctional (p)ppGpp synthetase II/guanosine-3′,5′-bis pyrophosphate 3′-pyrophosphohydrolase gene (spoT), enolase gene (eno), fructose-1,6-bisphosphatase 1 class 2 gene (glpX), fructose-1,6-bisphosphatase class 1 gene (fbp), GDP pyrophosphokinase/GTP pyrophosphokinase gene (relA), glucose-6-phosphate isomerase gene (pgi), glyceraldehyde-3-phosphate dehydrogenase gene (gapA), glyceraldehyde-3-phosphate dehydrogenase gene (gapC), glycine cleavage system H protein gene (gcvH), glycine decarboxylase gene (gcvP), HTH-type transcriptional regulator GntR gene (gntR), leucine-responsive regulatory protein gene (lrp), methylglyoxal synthase gene (mgsA), phosphogluconate dehydratase gene (edd), phosphoglycerate dehydrogenase gene (serA), phosphoglycerate kinase gene (pgk), ribulose-phosphate 3-epimerase gene (rpe), RNA polymerase-binding transcription factor gene (dksA), serine hydroxymethyltransferase gene (glyA), transaldolase A gene (talA), transaldolase B gene (talB), or a combination thereof.
